(19) United States
(12) Patent Application Publication
(10) Pub. No.: US 2024/0390031 A1
(43) Pub. Date: Nov. 28, 2024

(54) MULTI-PORTAL SURGICAL TOOLS AND SYSTEMS

(71) Applicant: Amplify Surgical, Inc., Irvine, CA (US)

(72) Inventors: Andy Wonyong Choi, Irvine, CA (US); Clark Hutton, Carlsbad, CA (US)

(21) Appl. No.: 18/674,784

(22) Filed: May 24, 2024

Related U.S. Application Data
(60) Provisional application No. 63/504,248, filed on May 25, 2023.
(60) Provisional application No. 63/611,874, filed on Dec. 19, 2023.

(51) Int. Cl. A61B 17/34; A61B 90/50

(52) U.S. Cl. CPC A61B 17/3423; A61B 90/50; A61B 2017/3443; A61B 2017/3447

(57) ABSTRACT

A multi-portal method for treating a subject's spine includes distracting adjacent vertebrae using a distraction instrument positioned at a first entrance along the subject to enlarge an intervertebral space between the adjacent vertebrae. An interbody fusion implant can be delivered into the enlarged intervertebral space. The interbody fusion implant can be positioned directly between vertebral bodies of the adjacent vertebrae while endoscopically viewing the interbody fusion implant using an endoscopic instrument. The patient's spine can be visualized using endoscopic techniques to view, for example, the spine, tissue, instruments, and implants before, during, and after implantation, or the like. The visualization can help a physician throughout the surgical procedure to improve patient outcome.

### CROSS-REFERENCE TO RELATED APPLICATIONS

The present application claims the benefit of U.S. Provisional Patent Application No. 63/504,248, filed May 25, 2023, and U.S. Provisional Patent Application No. 63/611,874, filed Dec. 19, 2023, the disclosures of which are incorporated herein by reference in their entireties.

### TECHNICAL FIELD

The present disclosure relates generally to medical systems and, more particularly, to systems, devices, and methods for performing multi-portal surgical procedures.

### BACKGROUND

Individuals often suffer from damaged or displaced spinal discs and/or vertebral bodies due to trauma, disease, degenerative defects, or wear over an extended period of time. One result of this displacement or damage to a spinal disc or vertebral body may be chronic back pain. A common procedure for treating damage or disease of the spinal disc or vertebral body may involve partial or complete removal of an intervertebral disc. An intervertebral implant (commonly referred to as an interbody spacer or cage) can be inserted into the cavity created where the intervertebral disc was removed to help maintain height of the spine and/or restore stability to the spine. An interbody spacer may also provide a lordotic correction to the curvature of the spine. An example of an interbody spacer that has been commonly used is a fixed dimension cage, which typically is filled with bone and/or bone growth-inducing materials. Unfortunately, it may be difficult to implant the interbody spacer at the intended implantation site between vertebral bodies. Additionally, conventional surgical techniques can cause a significant amount of trauma at or near the implantation site (e.g., injury to nerve tissue), which can significantly increase recovery time and/or lead to patient discomfort.

Spinal nerve compression can be caused by narrowing of the spinal canal associated with arthritis (e.g., osteoarthritis) of the spine, degeneration of spinal discs, and thickening of ligaments. Arthritis of the spine often leads to the formation of bone spurs, which can narrow the spinal canal and press on the spinal cord. In spinal disc degeneration, inner tissue of the disc can protrude through a weakened fibrous outer covering of the disc and can press on the spinal cord and/or spinal nerve roots. Ligaments located along the spine can thicken over time and press on the spinal cord and/or nerve roots. Unfortunately, spinal nerve compression can cause lower back pain, hip pain, and/or leg pain and may also result in numbness, depending on the location of the compressed nerve tissue. For example, spinal stenosis that causes spinal cord compression in the lower back can cause numbness of the legs. It is difficult to visualize internal tissue when removing tissue, often resulting in injury or removal of nerve tissue. Accordingly, there is a need for improved surgical systems, visualization techniques, and/or related technologies.

### BRIEF DESCRIPTION OF THE DRAWINGS

FIG. 1 is a side view of a multi-portal endoscopic surgical system in accordance with an embodiment of the disclosure.

FIG. 2A is a schematic top plan view showing surgical approaches to a lumbar spine for performing procedures.

FIG. 2B is an isometric view of the lumbar spine of FIG. 2A.

FIG. 3 is an isometric view of a multi-portal instrument holder in accordance with an embodiment of the disclosure.

FIG. 4 is a top view of the multi-portal instrument holder of FIG. 3.

FIG. 5 is a cross-sectional view of the multi-portal instrument holder taken along line 5-5 of FIG. 4.

FIG. 6 is a cross-sectional view of a multi-portal instrument holder in accordance with another embodiment of the disclosure.

FIG. 7 is an isometric view of a multi-portal instrument holder in accordance with another embodiment of the disclosure.

FIG. 8 is a top view of the multi-portal instrument holder of FIG. 7.

FIG. 9 is an isometric view of an ergonomic arm support in accordance with an embodiment of the disclosure.

FIG. 10 is a top view of the support of FIG. 9.

FIGS. 11A-11C are cross-sectional views of the support of FIG. 9 in various configurations.

FIG. 12 is an isometric view of an ergonomic support in accordance with an embodiment of the disclosure.

FIG. 13 is an isometric view of the support in a user-specific support configuration.

FIG. 14 is a cross-sectional view of the support of FIG. 12.

FIG. 15 is a side view of a multi-portal endoscopic surgical system with an articulatable instrument holder in accordance with an embodiment of the disclosure.

FIG. 16 is a side view of an instrument clamp of the instrument holder in accordance with an embodiment of the disclosure.

FIG. 17 shows the instrument holder of FIG. 16 holding an instrument.

FIG. 18 shows an instrument clamp of the instrument holder in accordance with another embodiment of the disclosure.

FIG. 19 shows the instrument clamp of FIG. 18 holding an instrument.

FIG. 20 illustrates a biasing instrument clamp in accordance with another embodiment of the disclosure.

FIG. 21 illustrates the clamp of FIG. 20 holding an instrument.

FIG. 22 is a plan view of a surgical kit in accordance with an embodiment of the disclosure.

FIGS. 23-25B illustrate surgical steps for performing spinal procedures in accordance with embodiments of the disclosure.

FIGS. 26A and 26B are side views of a multi-portal surgical system at different positions in accordance with an embodiment of the disclosure.

FIGS. 27A and 27B are superior-to-inferior views of a multi-portal surgical system at different positions in accordance with an embodiment of the disclosure.

FIG. 28 is an isometric view of a curved triangulation guide holding instruments in accordance with an embodiment of the disclosure.

FIG. 29 is a top view of the curved triangulation guide of FIG. 28.

FIGS. 30 and 31 are side views of the curved triangulation guide of FIG. 28.

FIG. 32 is an isometric view of a straight triangulation guide holding instruments in accordance with an embodiment of the disclosure.

FIG. 33 is a top view of the straight triangulation guide of FIG. 32.

FIGS. 34 and 35 are side views of the straight triangulation guide of FIG. 32.

FIG. 36 is an isometric view of an articulating triangulation guide in accordance with an embodiment of the disclosure.

FIG. 37 is a top view of the articulating triangulation guide in accordance with an embodiment of the disclosure.

FIGS. 38 and 39 are side views of the articulating triangulation guide of FIG. 36.

FIGS. 40 and 41 are side views of a joint assembly for the articulating triangulation guide of FIG. 36.

FIGS. 42 and 43 are side views of a multi-portal endoscopic surgical system in accordance with an embodiment of the disclosure.

### DETAILED DESCRIPTION

The following disclosure describes various embodiments of medical systems, devices, and associated methods of use. At least some embodiments of a surgical system provide intraoperative visualization capability. The system can include multi-portal instrument holders configured to hold multiple instruments, including cannulas, surgical instruments, cameras, combinations thereof, or the like. For example, the multi-portal instrument holders can hold cannulas while a series of instruments are delivered through the cannulas. The instruments can be used to alter tissue (e.g., shape, crush, separate, cut, debulk, break, fracture, or remove tissue), create working spaces, create delivery paths, prepare an implantation site, implant a device, combinations thereof, or the like. A visualization device can be positioned in one of the cannulas to provide viewing of a working space inside the patient. In some embodiments, the multi-portal instrument holders can hold an instrument (e.g., a visualization device) and a cannula while a series of instruments are delivered through the cannula. The multi-portal instrument holders can be unlocked to adjust, for example, the distance between the instruments, orientation between the instruments, depth of the instruments, etc. The multi-portal instrument holders can be locked to hold the instruments at, for example, a specific relative position with respect to each other, a position relative to the patient, etc. The system can also include one or more customizable physician pillows configured to assist with positioning the physician's body (e.g., hand(s), wrist, arm, etc.) during the surgical procedure. Physician-specific pillows can be configured preoperatively and/or intraoperatively and can be reusable or disposable. The physician-specific pillows can be selected based on the instrument configurations (e.g., configuration of surgical instruments, visualization instruments, etc.), surgical techniques to be used, period of support, contours of the patient's body, or the like.

Instrument and/or tissue visualization can help a physician identify issue, remove tissue under visualization, and/or prevent or limit injury or damage to non-targeted organs and tissues. In endoscopic-assisted surgeries, instruments and implantable devices can be precisely positioned using minimally invasive techniques to improve outcomes and reduce recovery times. Certain details are set forth in the following description and in the figures to provide a thorough understanding of such embodiments of the disclosure. Other details describing well-known structures and systems often associated with, for example, surgical procedures are not set forth in the following description to avoid unnecessarily obscuring the description of various embodiments of the disclosure.

### A. OVERVIEW

At least some embodiments are directed to multi-portal surgical systems configured to treat patients with, for example, nerve compression, damaged or displaced spinal features (e.g., spinal discs and/or vertebral bodies), or other conditions. For example, the surgical systems can be used to reduce or eliminate nerve compression, implant a fixed or expandable interbody device (e.g., devices to space apart vertebral bodies, restore stability of the spine, provide lordotic correction, etc.), perform discectomies, perform microdiscectomies, perform laminotomies, combinations thereof, or other surgical procedures. Multi-portal instrument holders can be configured to hold, for example, multiple cannulas, cannulas and surgical instruments, multiple surgical instruments, or the like. Physician positioners can help support or otherwise position the physician's body. Physician positioners can be pillows (e.g., foam cushion pillows, inflatable pillows, etc.) configured to support the user's hand, wrist, and/or arm during at least a portion of a surgical procedure. In decompression procedures, split cannulas can be used to access nerve compression sites. The multi-portal instrument holders can grip and hold the split cannulas and/or surgical instrument. The multi-portal holders can be coupled (e.g., coupled via an adhesive platform or base, articulating arm, etc.) to the patient's body, operating table, and/or other attachment feature to, for example, reduce, inhibit, or limit movement of the cannulas. Visualization instruments in the patient can provide viewing of working spaces, tissue contributing to the nerve compression, and the tissue removal instruments. Tissue can be safely removed under endoscopic visualization.

In some embodiments, a multi-portal instrument holder can be a triangulation guide having a guide body defining an elongated slot configured to receive multiple instruments. The guide body can be configured to allow sliding of the instruments along the slot while maintaining triangulation of the instruments. In some embodiments, maintaining triangulation of the instruments includes keeping the instruments at a triangulation relationship by, for example, keeping the instruments aligned or positioned along an imaginary plane, a reference line, etc. The configuration of the triangulation guide can be selected based on the desired triangulation of the instruments. The triangulation guide can limit movement of instruments to keep the instruments within a working space or region within the patient. The triangulation guide can limit the range of motion of the instruments to keep distal ends of the instruments along a target path or target region.

Any number of pillows can be used during the surgical procedure. For example, sets of pillows can be used during corresponding sets of surgical actions. For example, a first set of pillows can be used to perform a first decompression procedure at a first level of the patient's spine, a second set of pillows can be used to perform a second decompression procedure at a second level of the patient's spine, and a third set of pillows can be used to perform one or more implantation procedures at the first and/or second levels or other levels. The pillows can be coupled to the patient's body, operating table, and/or other attachments feature. Customizable ergonomic pillows can also be used to support the patient's body.

Endoscopic techniques can be used to view, for example, the spine (e.g., vertebral spacing, vertebral alignment, etc.), tissue (e.g., damaged or displaced sections of intervertebral cartilage disc, tissue contributing to nerve compression, etc.), instruments, and implants before, during, and after implantation, or the like. The visualization can help a physician throughout the surgical procedure to improve patient outcomes. In some embodiments, visualization instruments can be delivered through endoscopic cannulas (e.g., tubular closed cannulas, split cannulas, etc.). The cannulas can be held generally stationary or moved during one or more steps or the entire surgical procedure. During a surgical procedure, the cannulas can be positioned any number of times based on, for example, imaging, visualization of the surgical site, surgical steps to be performed, etc. In some procedures, the cannulas can be manually moved and may not be held by multi-portal instrument holders.

In some procedures utilizing multi-portal holders, the user can reposition the multi-portal holders to adjust the position of instruments. If the multi-portal holder is locked, the relative position of the instruments can be maintained. If the multi-portal holder is unlocked, the user can adjust the relative position between the instruments. This allows for flexibility when repositioning instruments. In some embodiments, instruments can move slightly with respect to one another to increase the range of motion of the instruments. The physician, nurse, or member of the surgical team can manually hold the cannulas or surgical instruments at any desired time to assist with positioning.

Access instruments can be selected based on the location of the working space. A physician body can be supported by one or more pillows. In some procedures, split cannulas of different lengths can be used to sequentially access and remove tissue. The sizes of the cannulas can be selected based on the location (e.g., depth) of the tissue, anatomical structures surrounding access paths and/or targeted tissue, and/or configuration of instrument(s). The configuration of pillows can be selected based on target position(s) of the cannulas. In some procedures, both tubular closed cannulas and split cannulas can be utilized. For example, a tubular closed cannula can prevent instruments from contacting tissue laterally adjacent to the cannula. The split cannula can allow the instrument to be moved laterally out of the cannula and into a large working space in the patient. As such, ends of instruments can be positioned in relatively large working spaces relative to an access port or incision in the skin (i.e., the incision can be significantly smaller than the size of the working space within the patient).

In some procedures, pillows can be used for a portion of a surgical procedure in which the user may want to keep an instrument (e.g., a viewing instrument, a surgical tool, etc.) at a stationary position for a relatively long period of time. The user may not use pillows for portions of the procedure in which the user plans to frequently reposition instruments. In some procedures, multiple pillows can be sequentially attached to the same location along the patient. Cleaning procedures can be performed to enhance adhesion between the pillows and the patient.

In some embodiments, multi-portal endoscopic techniques can be used to alter tissue at different locations along the spine. Bony features (e.g., facets and surrounding bone) of vertebrae can be removed to perform, for example, transforaminal procedures. The implantation site can be prepared by performing a discectomy, an interbody preparation procedure, or the like.

Multi-portal endoscopy-assisted methods can include performing at least a portion of a surgical procedure by using a first portal site. The first portal site can serve as a working portal for working instruments. At least a portion of the surgical procedure uses an endoscope positioned via a second portal site (e.g., a visualization portal) spaced apart from the first portal site. The spacing can be selected based on location and accessibility of the treatment site(s), whether along the spine or at another location. For example, the portals can be spaced apart to allow equipment (e.g., cannulas, endoscopes, working instruments, etc.) to be directed generally toward a working space within the subject.

In some decompression procedures, surgical steps can minimize or reduce pressure applied to nerve tissue and can include removing tissue contributing to stenosis, tissue pushing against nerve tissue, bulging sections of intervertebral cartilage disc, or the like. For example, tissue can be removed to enlarge an epidural space to reduce spinal cord compression.

In some aspects, techniques described herein relate to a multi-portal method for treating a subject. The methods include inserting a first distal end of a first split cannula into a first entrance formed in a subject. The first split cannula includes a first proximal end with a first flange configured to contact the subject's skin. A second distal end of a second split cannula can be inserted into a second entrance formed in the subject. The second entrance is spaced apart from the first entrance. The second split cannula includes a second proximal end with a second flange configured to contact the subject's skin. A distal end of an instrument positioned along a first passage of the first split cannula can be using a visualization instrument positioned along a second passage of the second split cannula.

In some aspects, the technology relates to a split cannula including a port flange and a split shaft connected to the port flange. The split shaft includes a tapered distal end configured to penetrate tissue to position the port flange proximate to a subject's skin. The split shaft also includes a plurality of spaced-apart motion inhibitors configured to contact tissue of a subject so as to inhibit movement of the split cannula relative to the subject.

In some aspects, the techniques described herein relate to performing a multi-portal spinal surgical procedure using first and second split cannulas. The multi-portal spinal surgical procedure can be a decompression procedure, an oblique lumbar interbody fusion (OLIF) procedure, a lateral lumbar interbody fusion (LLIF) procedure, a posterior lumbar interbody fusion (PLIF) procedure, a transforaminal lumbar interbody fusion (TLIF) procedure, an anterior lumbar interbody fusion (ALIF) procedure, or combinations thereof.

Embodiments of the present disclosure will be described more fully hereinafter with reference to the accompanying drawings in which like numerals represent like elements throughout the several figures, and in which example embodiments are shown. Embodiments of the claims may, however, be embodied in many different forms and should not be construed as limited to the embodiments set forth herein. The examples set forth herein are non-limiting examples and are merely examples among other possible examples.

### B. MULTI-PORTAL SURGICAL SYSTEMS

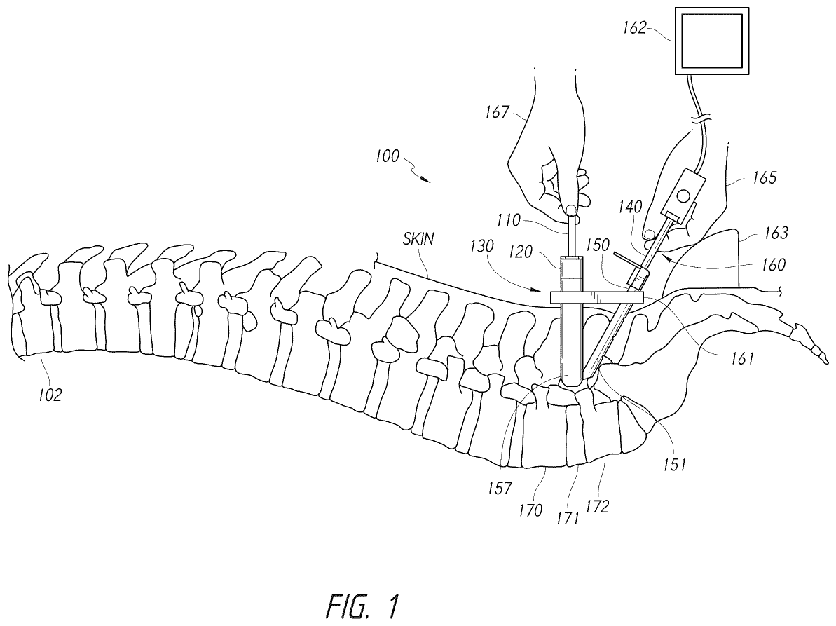

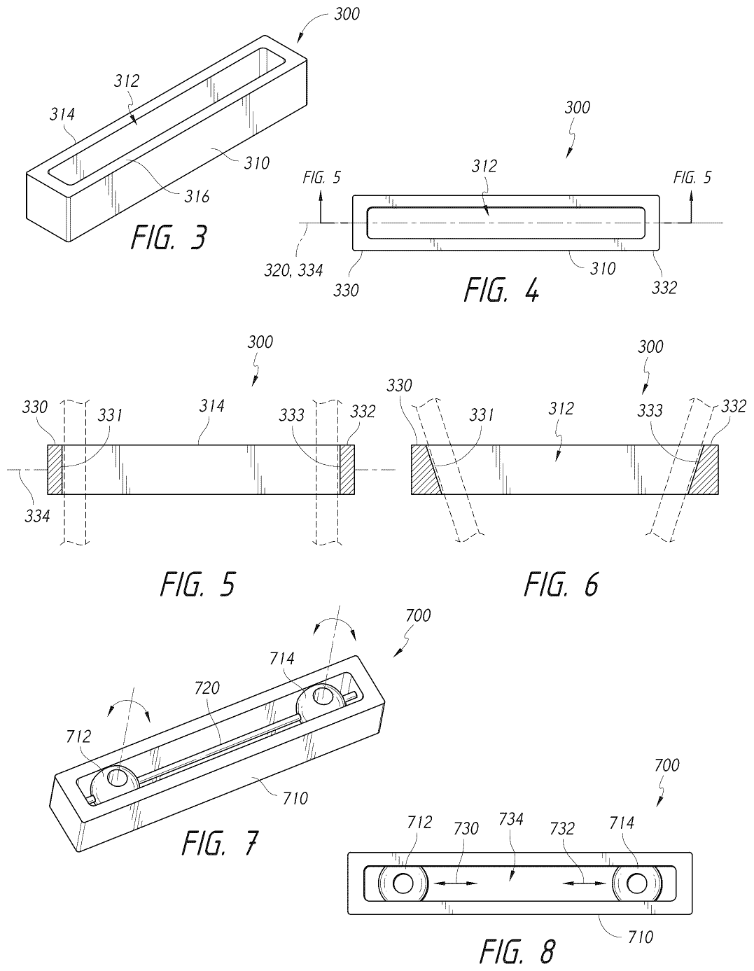

FIG. 1 is a side view of a spinal surgical system 100 (“system 100”) positioned along a human subject's spine 102 in accordance with an embodiment of the disclosure. The system 100 can include an instrument assembly 130, a visualization assembly 160, and a multi-portal instrument holder 161 (“instrument holder 161”). The instrument holder 161 can hold the instrument assembly 130 and visualization assembly 160. The instrument holder 161 can be configured to hold components (e.g., cannulas, surgical instruments, combinations thereof, etc.) of the instrument assembly 130, visualization assembly 160, and/or other equipment, such as surgical beds. Example features of multi-portal holders are discussed in connection with FIGS. 3-8.

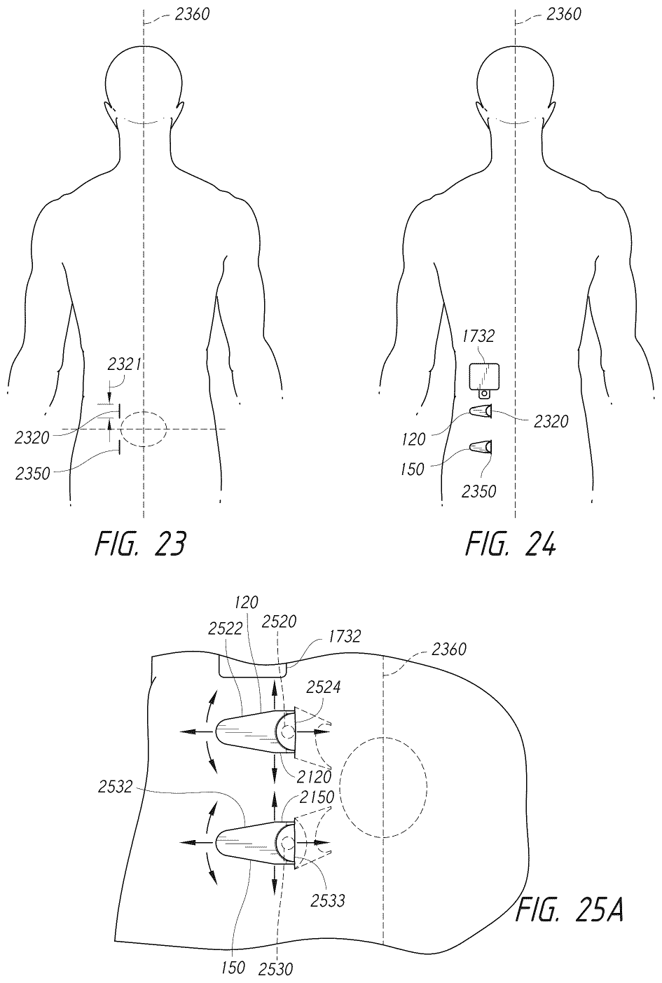

The instrument assembly 130 can include an instrument 110 and a cannula 120. The visualization assembly 160 can include a visualization instrument 140 and a cannula 150. The instruments 110, 140 can be moved distally and/or laterally out of the split cannulas 120, 150, which can be positioned in incisions or endoscopic ports, to access a relatively large working space along the patient's spine. The split cannulas 120, 150 can have longitudinally extending openings along their entire lengths or portion thereof (not visible in FIG. 1) to allow distal portions of the respective instruments 110, 140 to be moved laterally into and out of sides of the cannulas 120, 150. Positioning of instruments is discussed in connection with FIGS. 23-27B.

The illustrated cannula 150 has an open front side 151 (illustrated facing the inferior direction relative to the patient) through which the visualization instrument 140 can be moved. The cannula 120 has an open front side (not visible in FIG. 1) facing the subject's spine such that a backside atraumatic surface 157 contacts tissue to limit, reduce, or substantially eliminate trauma to tissue. A series of instruments can be delivered through the split cannula 120. In some procedures, the instrument 110 can be used to remove tissue (e.g., intervertebral disc 171, tissue contributing to stenosis, etc.), form access paths to implantation sites, prepare an implantation site by, for example, moving organs or tissue (e.g., moving nerve tissue), prepare vertebral bodies (e.g., roughening or shaping vertebral endplates), or the like. The instrument 110 can be removed and a distraction instrument (e.g., one or more dilators) can be delivered through the cannula 120 to distract adjacent vertebrae 170, 172, thereby enlarging the intervertebral space. An interbody implant can be delivered through the split cannula 120, or another cannula (e.g., a non-split tubular cannula), and into the enlarged intervertebral space. In expandable implant embodiments, an expandable interbody fusion implant can be expanded to push apart vertebral endplates.

With continued reference to FIG. 1, a customizable ergonomic pillow 163 can be used to position a user's body during the surgical procedure. The illustrated pillow 163 is configured to support the user's hand 165 holding the visualization assembly 160. Any number of pillows can be used during the surgical procedure to support the user's other hand 167 or other body parts. Additionally, sets of pillows can be used for corresponding sets for surgical actions. For example, a first set of pillows can be used to perform a first decompression procedure at a first level of the patient's spine, a second set of pillows can be used to perform a second decompression procedure at a second level of the patient's spine, and a third set of pillows can be used to perform one or more implantation procedures at the first and/or second level or other levels. The pillows can be coupled to the patient's body, operating table, and/or other attachment features. Example features of pillows are discussed in connection with FIGS. 9-14.

The visualization assembly 160 can provide intraoperative endoscopic viewing of workspaces, delivery paths, organs, tissue (e.g., nerve tissue) implantation sites, implants, interbody fusion devices (e.g., before, during, and/or after delivery), instrument(s) (including dispensers, dilators, decompression instruments, etc.), and other areas or features of interest. The position of the cannulas 120, 150 can be selected based on the procedure and optical characteristics (e.g., field of view, zoom capability, etc.) of the visualization assembly 160. The visualization assembly 160 can moved throughout the procedure to provide intraoperative endoscopic viewing of one, multiple, or all of the surgical steps. For example, the visualization assembly 160 can be used to view tissue contributing to nerve compression caused by narrowing of the spinal canal associated with arthritis of the spine, degeneration of spinal discs, and thickening of ligaments. Arthritis of the spine often leads to the formation of bone spurs, which can narrow the spinal canal and press on the spinal cord. This tissue can be viewed using the visualization assembly 160. In spinal disc degeneration, the visualization assembly 160 can view the inner tissue of the disc protruding through a weakened fibrous outer covering of the disc and pressing on the spinal cord and/or spinal nerve roots. The protruding tissue can be viewed before and/or during removal. The visualization assembly 160 can be used to also view ligaments pressing on the spinal cord and/or nerve roots to assist in treatment.

The visualization instrument 140 can be a low-profile fiber-optic endoscope positioned directly through an incision, an endoscopic port, or the like. The visualization instrument 140 can include one or more endoscopes having, without limitation, fiber optics (e.g., optical fibers), lenses, imaging devices, working lumens, light source controls, or the like for direct viewing or viewing via a display 162 (e.g., an electronic screen, a monitor, etc.). In some embodiments, the visualization instrument 140 can include a lumen through which fluid flows to irrigate the surgical site. For example, saline, or another suitable liquid, can be pumped through the visualization instrument 140 to remove tissue (e.g., loose tissue, bone dust, etc.) or other material impairing visualization. The visualization instrument 140 can also include one or more lumens (e.g., irrigation return lumens, vacuum lumens, etc.) through which the irrigation liquid can be withdrawn.

The visualization instrument 140 can illuminate the body cavity and enable high-resolution video visualization. A light source (e.g., a laser, light-emitting diode, etc.) located near or at the proximal end of the fiber optics can be used to transmit light to the distal end and provide illuminating light. This enables a surgeon to safely navigate into the subject's body and to illuminate specific body anatomy to view vertebral spacing, vertebral structures, nerves, bony buildup (e.g., buildup that could be irritating and pressing against nerves contributing to nerve compression), etc. In some embodiments, visualization optics for vision and illumination are included within the distal tip of the visualization instrument 140. The configuration and functionality of the visualization instrument 140 can be selected based on the desired field of view, viewing resolution, pan/zoom functionality, or the like. Irrigation techniques, visualization devices, instruments, cannulas, and visualization and surgical techniques are discussed in U.S. application Ser. No. 17/902,685 and U.S. application Ser. No. 16/687,520, which are incorporated by reference in their entireties.

To position the cannulas 120, 150, the cannulas can be inserted into entrances formed in the subject's skin. The multi-portal instrument holder 161 can be adjusted to hold the cannulas 120, 150 at the fixed or altered positions while instruments (e.g., instruments 110, 140) are delivered through the cannulas 120, 150. The instrument holder 161 can be used to set the distance between the cannulas 120, 150 and can be locked to hold the cannulas at, for example, a set distance and/or angular orientation. For example, the instrument holder 161 can have locking mechanisms that are locked by the user to hold the cannulas 120, 150 stationary relative to one another. The instrument holder 161 can be unlocked to reposition the cannulas. This process can be performed any number of times to reposition the cannulas 120, 150.

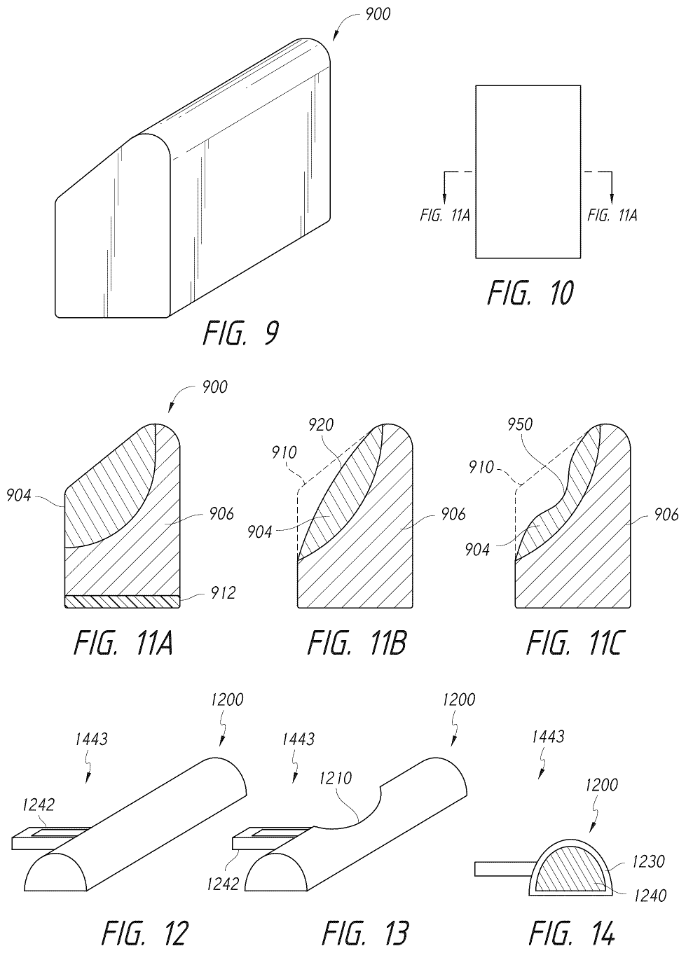

The instrument holder 161 can also be used to hold the instruments 110, 140 in a similar manner. In some embodiments, an instrument holder or cannulas 120, 150 and another multi-portal instrument holder holds the instruments 110, 140. This allows for flexibility during the surgical procedure to hold various components stationary relative to one another when desired. For example, a multi-portal instrument holder in the form of a triangulation guide can be used with the instruments 110, 140. A multi-portal instrument holder (e.g., instrument holder 700 of FIGS. 7 and 8) can hold the cannulas 120, 150 stationary relative to one another. This allows for some instruments to be moved relative to one another while other instruments are held relatively stationary.

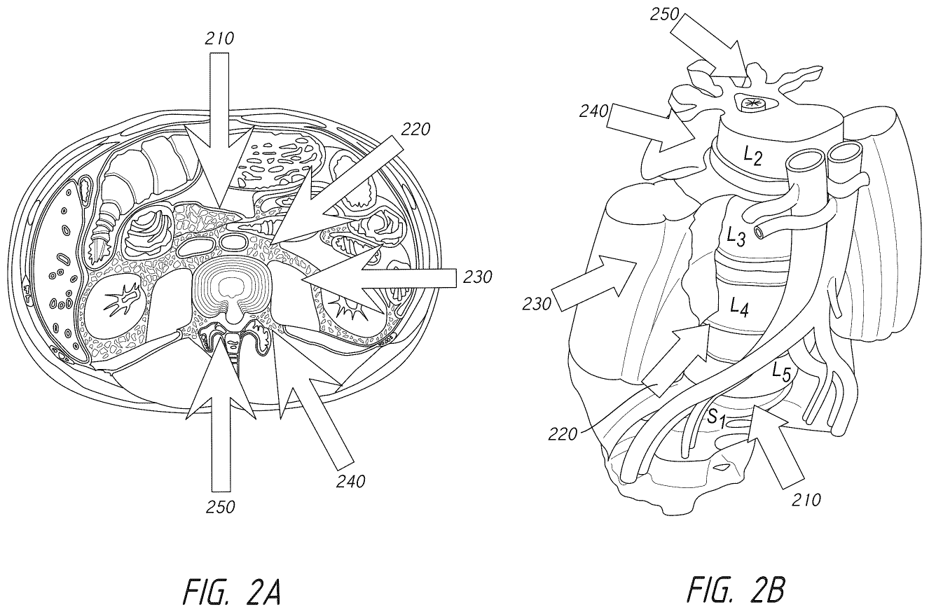

FIG. 2A is a schematic top plan view along the lumbar spine of a human subject and illustrates example approaches for performing procedures suitable for the system 100 of FIG. 1 and other systems disclosed herein. FIG. 2B is an isometric view of the lumbar spine of FIG. 2A. The number and configuration of physician-support pillows can be selected based on the spinal approach.

Referring to FIGS. 2A and 2B, surgical equipment can be delivered via different paths, including an ALIF path 210, an OLIF path 220, an LLIF or extreme lateral lumbar interbody fusion (XLIF) path 230, a TLIF path 240, and a PLIF path 250. These paths can also be used to perform other procedures disclosed herein. For example, one or more of the paths 210, 220, 230, 240, 250 can be selected for multi-portal endoscopic approaches to perform a wider array of lumbar spine procedures than conventional one-portal techniques. Cannulas can be positioned along the same path or different paths to allow for independent positioning and manipulation of the endoscopic camera of the surgical instruments, thereby providing greater flexibility and enhanced visualization of spinal anatomy.

Surgical instruments can remove tissue to define working space(s) inside the patient. In one example TLIF procedure, the transforaminal path 240 may be employed to implant a single small expandable or non-expandable interbody spacer at the intervertebral space. In one example PLIF procedure, two interbody spacers can be delivered along the posterior path 250 and implanted at the intervertebral space. The two interbody spacers can cooperate to keep the vertebral bodies at the desired spacing and may be larger than the TLIF spacer. Additionally, multiple interbody spacers can provide lordotic correction by providing support at different heights. In one example LLIF procedure, a single, relatively large interbody spacer can be delivered along the lateral path 230 and implanted to provide asymmetrical support. In one example ALIF procedure, an asymmetric interbody spacer can be delivered along the anterior path 210 to provide support consistent with lordosis at that portion of the spine. Lateral approaches, transforaminal approaches, and anterior approaches can be used to access the cervical spine, thoracic spine, etc. The number of instruments, configurations of instruments, implants, and surgical techniques can be selected based on the condition to be treated.

Referring now to FIG. 3, a multi-portal instrument holder 300 can be configured to hold multiple instruments. The instrument holder 300 can assist with positioning of instruments by, for example, limiting movement of the instruments (e.g., movement relative to one another, movement relative to the patient, etc.), providing guided movement, or the like. The instrument holder 300 can include a guide body 310 and an opening or slot 312 (“slot 312”). The guide body 310 can include sidewalls 314, 316 positioned on either side of the slot 312. The slot 312 can be a linear slot (illustrated), a serpentine slot, or a slot shaped to define an instrument path. In some embodiments, the guide body 310 is configured to allow instruments to slide along the slot 312 while keeping the instruments positioned along, for example, an imaginary plane (e.g., imaginary plane 320 of FIG. 4), within the acceptable range of motion, or the like. The sidewalls 314, 316 can be configured to slidably contact the instruments and can include, without limitation, one or more positioning-assist features (e.g., notches, labels, grooves, magnets, or combinations thereof). The positioning-assist features can help a user determine the location of the instrument based upon tactile feedback, visual inspection, or the like.

The instrument holder 300 can be made, in whole or in part, of one or more polymers, composites, plastics, metals, or materials suitable for contacting instruments. In some embodiments, the slot 312 can be lined with a compressible material to provide cushioning of instruments. This may allow for an increased range of motion of the instruments, including a slight amount of out-of-plane rotation, thereby providing for position flexibility to adjust the relative position, including distance, angular position, etc., of the distal ends of the instruments. The configuration of the instrument holder 300 can be selected based on the procedure to be performed.

Referring now to FIG. 4, the elongated slot 312 can extend between ends 330, 332 of the guide body 310. A longitudinal axis 334 of the instrument holder 300 can be generally aligned with the imaginary plane 320. The instruments can be translated along the longitudinal axis 334 and can be rotated along the imaginary plane 320. The configuration of the elongated slot 312 can be selected based on, for example, the configuration of instruments to be held, range of motion of the instruments, etc.

FIG. 5 is a cross-sectional view of the instrument holder 300 taken along line 5-5 of FIG. 4 in accordance with an embodiment of the disclosure. The ends 330, 332 can include generally vertical sidewalls 331, 333 to allow instruments (two illustrated in phantom line) to be positioned generally orthogonal to the longitudinal axis 334 of the instrument holder 300. The configuration of the ends 330, 332 can be selected to limit the range of motion of the instruments. For example, FIG. 6 illustrates an embodiment of the instrument holder 300 with ends 330, 332 with slanted surfaces or sidewalls 331, 333 configured to angle the instruments (two instruments illustrated in phantom line) toward one another. The angular orientation and position of the sidewalls 331, 333 can be selected based on the desired positioning of the instruments located at ends of the slot 312.

FIG. 7 is an isometric view of a multi-portal instrument holder 700 in accordance with another embodiment of the disclosure. FIG. 8 is a top view of the instrument holder 700. Referring now to FIG. 7, the instrument holder 700 can be configured to pivotally hold multiple instruments and can include a holder body 710 and pivoting sliders 712, 714. The holder body 710 can include one or more optional tracks 720 along which the sliders 712, 714 can move. The instruments can be moved away from or toward one another while maintaining triangulation of the instruments. The description of one slider 712, 714 applies to the other slider 712, 714 unless indicated otherwise.

The sliders 712, 714 can be replaced with other sliders configured to hold additional instruments. This allows for interchangeability of sliders to reconfigure the instrument holder 700 to, for example, clamp onto various instruments, provide range of motions, or the like. For example, one slider 712, 714 can provide for translation and pivoting of an instrument, and the other slider 712, 714 can hold an instrument translationally fixed while allowing for pivoting of such instrument. In some embodiments, the sliders 712, 714 include, without limitation, one or more pivoters, locking mechanisms, retainers, or the like. In some embodiments, the slider 712 can include a pivoter configured to allow rotation of a retained instrument relative to an instrument retained by the slider 714. In some embodiments, the slider 712 can include a locking mechanism configured to lock the retained instrument at, for example, a position, an angular orientation, or the like.

FIG. 8 shows the sliders 712, 714 configured to move along a slot 734, as indicated by arrows 730, 732. The configuration of the holder body 710, number of sliders, range of motion of the sliders, configuration of the sliders, and slider features can be selected based on the procedure to be performed. Sliders can be incorporated into other instrument holders discussed herein, including instrument holder 161 of FIG. 1 and instrument holder 300 of FIGS. 3-6.

FIG. 9 is an isometric view of a customizable ergonomic support 900 in accordance with an embodiment of the disclosure. FIG. 10 is a top view of the support 900 of FIG. 9. FIGS. 11A-11C are cross-sectional views of the support 900 taken along line 11A-11A of FIG. 10 in accordance with various embodiments of the disclosure. Referring now to FIGS. 9 and 10, the support 900 can be a customizable pillow that can assist with positioning of the physician's body. The support 900 can be a pillow and can include, without limitation, one or more moldable materials, thermoformable materials, thermosetting materials, removable sections, inflatable sections, or materials for customization. The materials can have (i) a forming state including at least one of a flowable state, an uncured state, or a thermoforming state, and (ii) a formed state including at least one of a non-flowable state, a cured state, or a thermoformed state.

FIG. 11A shows the pillow 900 with a mounting feature comprising an optional adhesive pad 912. The adhesive pad 912 can be adhered to a patient's skin, a surgical table, or another suitable mounting feature. Other mounting features can be coupled to or incorporated into the pillow 900.

Referring now to FIGS. 11A-11C, the pillow 900 can be customized by the user. The pillow 900 can have an initial configuration of FIG. 11A and a customized configuration, as shown in FIGS. 11B and 11C. The initial position 910 (illustrated in phantom line in FIGS. 11B and 11C) shows example amounts of temporary or permanent contouring. The pillow 900 can include a moldable region 904 and a non-formable support region or portion 906. The moldable region 904 can include, for example, one or more thermoformable materials, thermosetting materials, curable materials, foams (e.g., closed cell foam, open cell foam, etc.), inflatable members, or the like. The moldable region 904 can be comprised of a material that is more compressible than material of the support portion 906. This allows for local deformation to enhance comfort of the user while the support portion 906 provides stability relative to the underlying patient. FIG. 11B shows the moldable region 904 having a convex region 920 along which the user's hand or wrist can roll. FIG. 11C shows the moldable region 904 having a concave region 950 for holding the user's hand or wrist generally stationary. For example, the concave region 950 can receive the backside of the user's hand or wrist. The moldable region 904 can be configured to be molded to the user's body such that the surgical support pillow comprises a user-specific, disposable surgical support pillow.

FIG. 12 is an isometric view of a customizable pillow 1200. FIG. 13 shows the customized pillow 1200 having a depression or recessed region 1210 configured to receive the user's arm or wrist. The pillow 1200 can support the user's arm or wrist while the pillow 900 of FIGS. 9-11C supports the user's hand.

Pillows can be coupled to one or more instrument holders. FIGS. 12-14 show the pillow 1200 coupled to a multi-portal instrument holder 1242. The multi-portal instrument holder 1242 can hold an instrument stationary relative the pillow 1200. In some embodiments, the multi-portal instrument holder 1242 is detachably coupled to the pillow 1200 by one or more pins, fasteners, snaps, etc. The allows replacement of the multi-portal instrument holder 1242. In some embodiments, the multi-portal instrument holder 1242 is integrated into or permanently coupled to the pillow 1200. The number of instrument holders, positions of the instrument holders, and configuration of the instrument holding pillow assembly 1443 can be selected based on the procedure to be performed. The multi-portal instrument holder 1242 can be an instrument holder and include features discussed in connection with FIGS. 1-11C.

FIG. 14 is a cross-sectional view of the pillow 1200 including a covering 1230 and an internal body 1240. The internal body 1240 can include one or more layers, inflatable members, mechanically expandable members, or the like. The configuration and number of features of the pillow 1200 can be selected based on the desired customization.

The pillows can be formed preoperatively or intraoperatively. In preoperative forming, a user can place a pillow on the user. The pillow can include a sterile covering or a bag (e.g., bag or covering 1230 of FIG. 14) suitable for contacting the patient. Once a pillow is positioned on a patient, a user can press on the pillow to form the pillow to the desired configuration. The formed pillow can then be made rigid by, for example, a drying process, curing process, cooling process, or other process setting the formed configuration. For example, if the pillow is a thermoformable pillow, the pillow can be heated to allow for thermoforming. Once shaped, the pillow can be cooled to permanently set the shape of the pillow. In some embodiments, the customized pillow can be removed from the patient and sterilized using a sterilization procedure. The sterilized pillow can then be directly applied to the patient. In other embodiments, the pillow can be placed in a sterilized covering and then positioned on the patient.

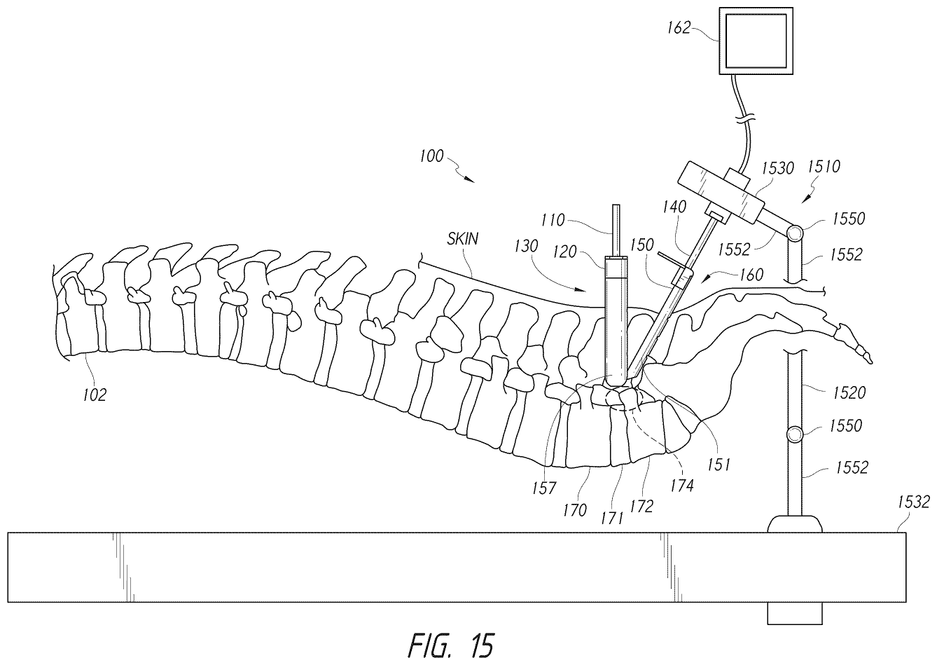

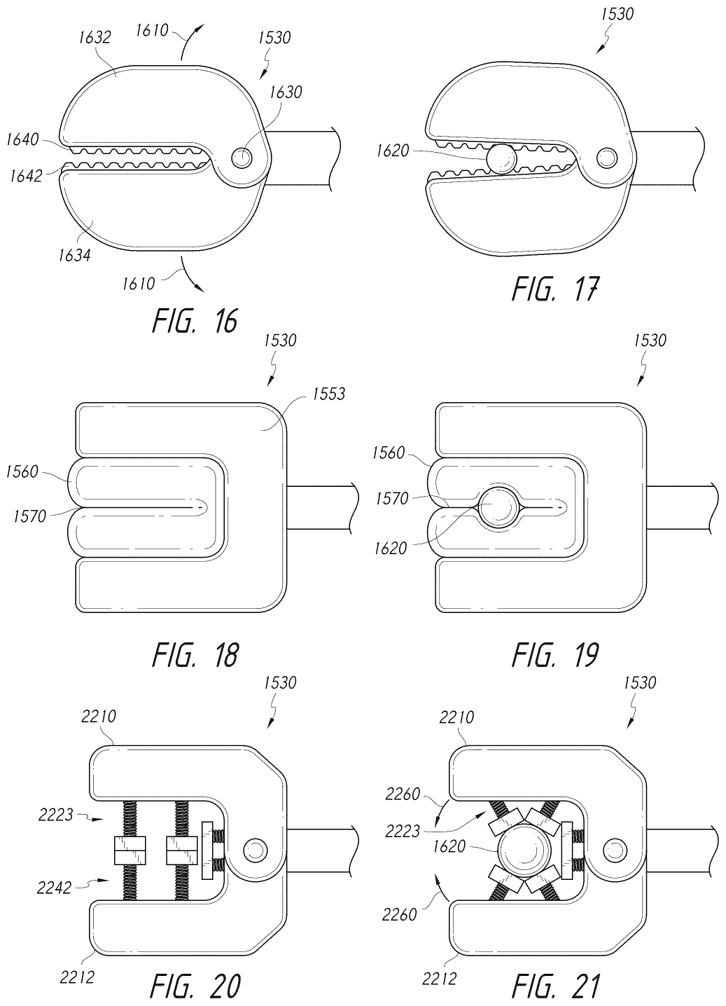

FIG. 15 is a side view of the multi-portal endoscopic surgical system 100 in accordance with another embodiment of the disclosure. The system 100 of FIG. 15 is generally similar to the system 100 of FIG. 1 except as detailed below. The system 100 can include an instrument holder 1510 having an arm 1520 configured to be coupled to a structure (e.g., surgical bed, a mounting bar, etc.) and a clamp or jaw 1530 (“jaw 1530”) coupled to the arm 1520. The jaw 1530 is configured to grip the visualization instrument 140 when the arm 1520 is at a fixed configuration, thereby holding the visualization instrument 140 stationary relative to a patient. The arm 1520 can be fixedly coupled to a surgical bed 1532 by, for example, one or more clamps. The arm 1520 can include one or more joints (e.g., manual joints, motorized joints, etc.), linkages, motors, controllers, sensors, or the like. The configuration of the arm 1520 can be selected based on the desired range of motion, degrees of freedom, or the like. In some embodiments, the arm 1520 includes multiple joints 1550 and linkages 1552. The number of linkages, joints, and other components of the arm 1520 can be selected based on the desired positioning of the instrument and range of motion. Example jaws 1530 are discussed in connection with FIGS. 16-21.

FIGS. 16 and 17 show the jaw 1530 movable (indicated by arrows 1610 in FIG. 16) between an open configuration for receiving an instrument and then moved to a closed configuration (shown in FIG. 17) to hold an instrument 1620. The jaw 1530 can include a joint 1630 coupling an upper jaw 1632 to a lower jaw 1634. The upper and lower jaws 1632, 1634 can include retention members 1640, 1642 in the form of, for example, one or more teeth, compressible members, serrated portions, or the like. When the jaw 1530 is moved to a closed configuration, the instrument 1620 (FIG. 17) can be held by the retention members 1640, 1642.

FIGS. 18 and 19 show the jaw 1530 having a fixed configuration. The jaw 1530 includes a body 1553 and a compressible member 1560 with a slot 1570 for receiving an instrument. FIG. 19 shows an instrument 1620 that has been slid into the slot 1570. The compressible member 1560 applies pressure to hold the instrument 1620. A user can move the instrument 1620 relative to the body 1553 by overcoming the biasing force provided by the compressible member 1560. The compressible member 1560 can be made, in whole or in part, of one or more compressible materials, compliant materials, or the like. For example, the compressible member 1560 can be made, in whole or in part, of silicone, rubber, foam (e.g., open-cell foam, closed-cell foam, etc.), or other suitable compliant material. In some embodiments, the compressible member 1560 can include multiple layers each having different mechanical properties. This allows for customization of the jaw 1530.

Referring to FIGS. 20 and 21, the jaw 1530 includes spring-loaded grippers 2223 facing an opening of the jaw. The spring-loaded grippers 2223 can cooperate to hold an instrument 1620, as shown in FIG. 21. The spring-loaded grippers 2223 can include, without limitation, one or more springs (e.g., helical springs, leaf springs, etc.), inflatable members, plates, teeth, or other features. In some embodiments, the jaw 1530 can include an upper and lower jaw 2210, 2212 that can be moved between an open configuration for receiving an instrument and a closed configuration for retaining the instrument. In operation, the instrument 1620 can then be inserted into the opening 2242 of the jaw 1530. The upper and lower jaws 2210, 2212 can then be rotated toward one another, as indicated by arrows 2260 of FIG. 21 to close the open jaw. The spring-loaded grippers 2223 can be brought into contact with the instrument 1620 and can then be biased against the instrument 1620 to inhibit, limit, or substantially prevent movement of the instrument 1620.

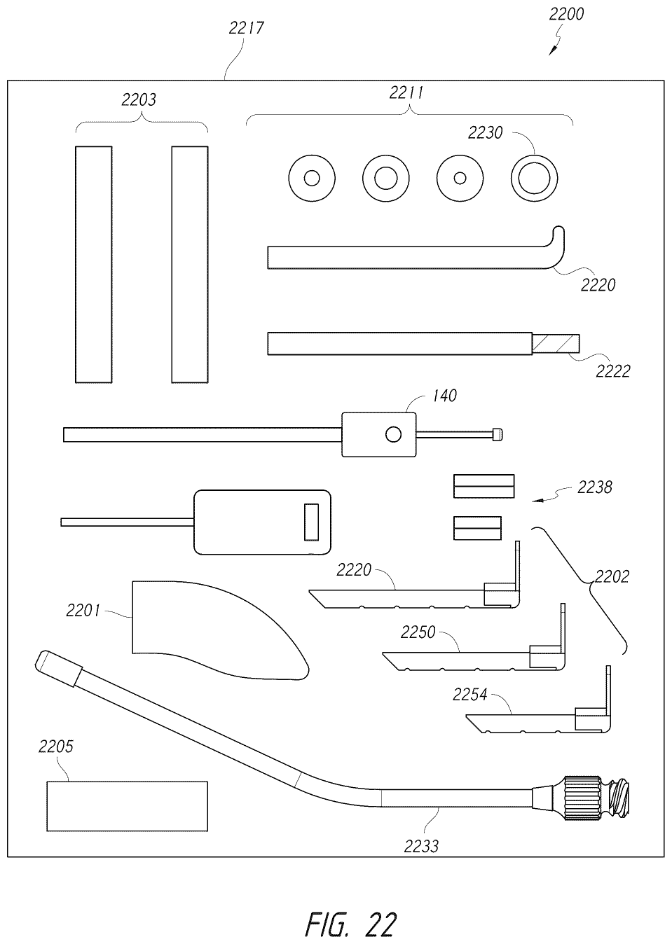

FIG. 22 is a top plan view of a surgical kit 2200 that includes components discussed in connection with FIGS. 1-21. The kit 2209 can include one or more pillows 2201, multi-portal instrument holders 2205, a set 2202 of split cannulas, tubular closed cannulas 2203, a set 2211 of ports, agent dispenser 2233, and implants 2238. A physician can select appropriate cannulas based on the entrance sites. In the illustrative embodiment, the set 2202 includes three cannulas 2220, 2250, 2254. A higher or lower number of cannulas can be provided and can be of the same or different sizes. For example, the cannulas 2220, 2250, 2254 can have different lengths to provide flexibility to access internal sites. In some embodiments, portions of two or more of the cannulas 2220, 2250, 2254 can be geometrically congruent. This allows for consistent usage of different instruments. For example, split shafts of cannulas can be geometrically congruent to provide for similar interaction of instruments.

The kit 2200 can further include a plurality of decompression instruments. In the illustrated embodiment, the kit 2200 includes a debulking instrument 2222 and a reamer 2222. If the decompression instruments are utilized, a physician can select the port 2230 with a large opening. The kit 2200 can also include scalpels, dilators, rongeurs, or other surgical instruments. The kit 2200 can include components of, or the entire, visualization instrument 140, the delivery or deployment instrument, and implantable devices 2238. The configuration and components of the kit can be selected based upon the procedure to be performed. Moreover, one or more of the kit's components can be disposable and can be made from metal, polymer, ceramic, composite, or other biocompatible and sterilizable material. The kit 2200 can include a container 2217 for holding the components. The container 2217 can be a reusable or disposable box. The multi-portal instrument holders 2205 can be configured to couple together cannulas (e.g., cannulas 2220, 2250, 2254), instruments (e.g., instruments 2220, 2222, etc.), or the like.

In operation, a user can select tools based on the location of the working space. In some procedures, cannulas of different lengths can be used to sequentially access and remove tissue. The cannula configuration can be selected based on the location (e.g., depth) of the tissue, anatomical structures surrounding access paths and/or targeted tissue, and/or configuration of instrument(s). In some procedures, both tubular closed cannulas and split cannulas can be utilized. The split cannula can allow the instrument to be moved laterally out of the cannula into a large working space in the patient. As such, instruments can be positioned in relatively large working spaces relative to an access port or incision in the skin (i.e., the incision can be significantly smaller than the size of the working space within the patient). The pillow 2201 can be formed by simulating usage of kit components that will be used in the procedure.

Systems, components, and instruments disclosed herein can be disposable or reusable. For example, components of the kit 2200 can be disposable to prevent cross-contamination. As used herein, the term “disposable” when applied to a system or component (or combination of components), such as a cannula, port, dispenser, instrument, tool, or a distal tip or a head (e.g., a reamer head, a rongeur, etc.), is a broad term and generally means, without limitation, that the system or component in question is used a finite number of times and is then discarded. Some disposable components are used only once and are then discarded. In other embodiments, the components and instruments are non-disposable and can be used any number of times. The cannulas, dispenser 2233, and other kit components can be reusable or disposable and configured to be used with one another.

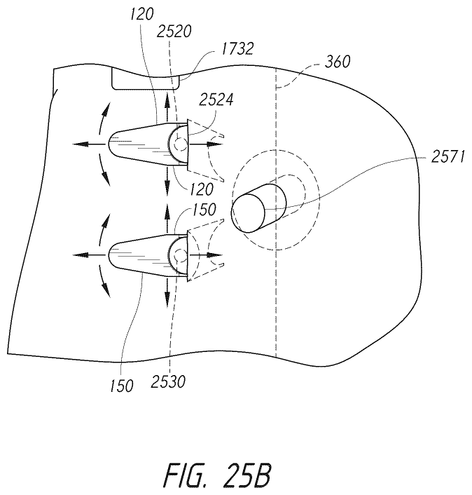

FIG. 23 shows two incisions 2320, 2350 for accessing the left side of a lumbar spine. FIG. 24 shows split cannulas 120, 150 positioned in the incisions 2320, 2350, respectively, for instrument insertion. The pillow 1732 can be positioned adjacent to the cannulas 120, 150. For example, the illustrated pillow 1732 is positioned adjacent to the cannula 120. The user's left hand can be supported by the pillow 1732 while gripping an instrument (e.g., instrument 2520 of FIG. 25A) for longer procedures. Additional pillows can be positioned along the patient to provide support when using the instrument 2530 of FIG. 25A.

The cannulas 120, 150 can be angled toward each other, as shown in FIG. 1, while maintaining a minimum distance of separation. Additional cannulas can be inserted into the subject to access other regions and/or provide alternative access paths. The number of cannulas can be selected based on the procedure to be performed, number of concurrently utilized working instruments, instrument trajectories, or the like. In some multi-portal procedures, a single cannula is positioned in the patient while visualization instruments or working instruments inserted directly into the patient. Cannulas can be inserted into, removed from, and/or replaced any number of times during the procedure. In some embodiments, one or more steps of the procedure (or the entire procedure) are performed without utilizing any cannulas. In steps or procedures without cannulas, access devices can be positioned in incisions to inhibit or limit tearing of skin or shallow tissue. The access devices can be ports, including flanged ports.

FIG. 25A shows instruments 2520, 2530 (illustrated in dashed line) positioned in U-shaped passages or channels of the cannulas 120, 150. The cannulas 120, 150 and instruments 2520, 2530 can be moved together or independently, as in indicated by arrows. Tissue 2524, 2533 extending across the open sides of the cannulas 120, 150 helps keep portions of the instruments 2520, 2530 within the proximal ends of the cannulas 120, 150. The cannulas 120, 150 can include insertion stops, illustrated as outwardly extending flanges 2522, 2532, respectively, configured to contact the patient's skin or multi-portal instrument holder, such as instrument holders discussed in connection with FIGS. 1 and 3-8. The cannulas 120, 150 and instruments 2520, 2530, respectively, can be positioned any number of times. Additionally or alternatively, additional incisions can be made along the patient to reposition the cannulas 120, 150 or to insert additional cannulas and instruments.

FIG. 25B shows a tubular cannula 2571 positioned to deliver a spinal implant, such as an intervertebral cage, interspinous spacer, screws, etc. The tubular cannula 2571 can also be positioned on the opposite side of the sagittal plane 360 or at other locations. Instruments, implants, and other items can be delivered through the tubular cannula 2571.

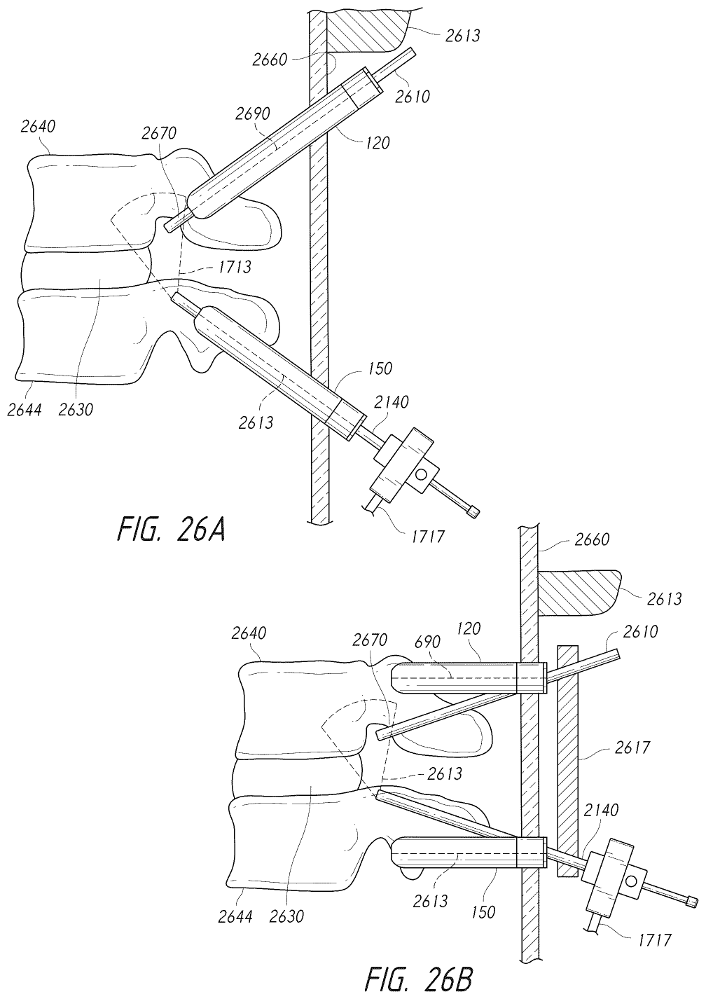

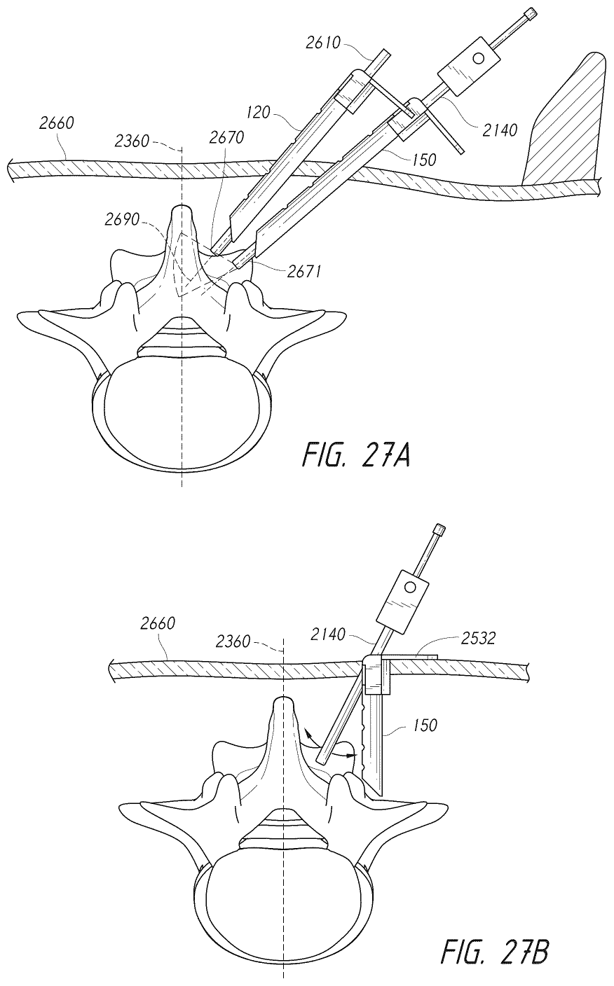

The instruments 2520, 2530 of FIGS. 25A and 25B can be the same as or similar to the instruments 110, 140 of FIG. 1 or other instruments disclosed herein, including instruments disclosed in U.S. application Ser. No. 17/902,685 and U.S. application Ser. No. 16/687,520. Details of example surgical procedures are discussed in connection with FIGS. 26A-27B.

### C. SURGICAL TECHNIQUES

FIGS. 26A and 26B are side views of multi-portal surgical systems at different positions in accordance with an embodiment of the disclosure. The cannulas 120, 150 extend through the subject's skin 2660 (thickness not illustrated at scale) at different locations and orientations. The visualization instrument 140 has a field of view 1713 suitable for viewing the spinal column and can be positioned using, for example, a transforaminal approach, a posterior approach, or a lateral approach. A pillow 2613 can be positioned along the patient. An instrument holder 1717 can hold the visualization instrument 140 at a desired position for visualization and the instrument holder 2617 (FIG. 26B) is spaced apart from the pillow 2613. In some embodiments, the instrument holder 2617 (FIG. 26B) is coupled to the pillow 2613, as discussed in connection with FIGS. 12-14.

To allow significant instrument movement, the cannulas 120, 150 can have axial lengths shorter than a distance from the incision in the skin 2660 to the spine. The sizes of the cannulas 120, 150 can be selected based on the size and configuration of the incision and characteristics of the tissue. For example, the enlarged port body of the cannula can be sufficiently long to extend through the subject's skin, fascia, and muscle. The channel of the cannulas can be sufficiently large to allow instruments to be inserted into and distally along the channel, which can prevent or inhibit tearing of tissue. The tissue can cover the channel to keep at least the proximal position of the instrument in the cannula. Instruments can have relatively small diameters relative to a width of sidewall openings of the cannulas to limit or inhibit tearing of the tissue around the incision. In some procedures, ports can be installed in some incisions and cannulas can be installed in other incisions without ports. A physician can determine whether to install ports based on the instruments to be utilized and the position of the incisions. Cannulas, ports, and other components can be installed in each of the incisions.

Referring to FIGS. 26A and 26B together, the visualization instrument 2140, which is outside intervertebral spaces, is positioned to view at least a portion of an intervertebral disc 2630, vertebral bodies 2640, 2644, and/or the distal portion 2670 of the instrument 2610. Fluoroscopy, MR imaging, CT imaging, direct visualization, or other visualization techniques can be used in addition to or in lieu of the endoscopic viewing. Additional instruments can be sequentially delivered through the cannula 120. In some procedures, multi-modality imaging of the target site can be performed using an external imaging device and the visual visualization instrument 2140. The intraoperative imaging can be displayed via one or more digital screens (e.g., endoscopic imaging and fluoroscopy on different screens) in the surgical room.

FIG. 26A shows the cannulas 120, 150 angled toward a vertebral level to allow instruments 2140, 2610 to be kept generally aligned with the respective cannulas 150, 120. FIG. 26B shows the cannulas 120, 150 kept generally perpendicular to the subject's spine and the instruments 2140, 2610 are angled toward a vertebral level. The cannulas 120, 150 can be manually moved between the positions of FIGS. 26A and 26B. The multi-portal instrument holder 2617 holds the instruments 2140, 2610 in FIG. 26B.

FIGS. 27A and 27B are superior-to-inferior views of the multi-portal surgical system at different positions in accordance with an embodiment of the disclosure. Open sides of one or both cannulas 120, 150 can face the subject's midsagittal plane 2360. The distal ends of the cannulas 120, 150 can be moved relative to the spine while the proximal ends of the cannulas 120, 150 remain generally stationary (e.g., at a fixed axial position relative to the skin 2760). Distal portions of the instruments 2670, 2671 can extend out of longitudinally extending openings of the cannulas 120, 150 to manipulate the tissue at the target site while shallow tissue (e.g., skin 2660) retains the respective cannulas 120, 150.

The length of the incisions can be selected to help inhibit or limit axial rotation of the cannulas. A ratio of the length of the incision to an outer width of the cannula can be less than or equal to, for example, 1.1, 1.2, 1.3, 1.4, or 1.5. For example, a ratio of a length 2321 of the incision 2320 of FIG. 23 to a transverse width split shaft width can be equal to or less than 1.5 to inhibit or prevent 90 degrees of rotation of the cannula 120 about its longitudinal axis 2690 (FIG. 27A). This keeps the open side of the cannula 120 generally facing the working space (e.g., working space along the subject's spine in FIGS. 26A and 26B). Instrument holders can hold the cannulas 120, 150, instruments 2610, 2140, or a combination of cannulas and instruments. The instrument holders can be particularly useful when performing surgical techniques involving three or more ports.

Instruments can be selected to treat, without limitation, spinal nerve compression (e.g., spinal cord compression, spinal nerve root compression, or the like), spinal disc herniation, osteoporosis, stenosis, or other diseases or conditions. After accessing the work space, the tissue removal tip can remove unwanted tissue, including, without limitation, tissue bulging from discs, bone (e.g., lamina, lateral recesses, facets including the inferior facets, etc.), bone spurs (e.g., bone spurs associated with osteoarthritis), tissue of thickened ligaments, spinal tumors, displaced tissue (e.g., tissue displaced by a spinal injury), or tissue that may cause or contribute to spinal nerve compression. The instrument, as well as other instruments (e.g., rongeurs, debulkers, scrapers, reamers, dilators, etc.), can be used to perform one or more dilation procedures, decompression procedures, discectomies, microdiscectomies, laminotomies, or combinations thereof. In procedures for treating stenosis, the instrument can be used to remove tissue associated with central canal stenosis, lateral recess stenosis, and/or other types of stenosis. In some decompression procedures, the instrument can be a tissue removal device used to, for example, remove bone, separate the ligamentum flavum from one or both vertebrae, cut or debulk the ligamentum flavum, remove loose tissue, and remove at least a portion of the intervertebral disc. Each stage can be performed with a different instrument.

The visualization instrument 2140 of FIGS. 26A-27B can be, without limitation, an endoscopic instrument that includes fiber optics suitable to image the treatment site and surrounding tissues, such as the spinal cord, nerves branching from spinal cord, ligaments, vertebrae 2640, 2644, intervertebral disc 2630, or any other features or anatomical structures of interest while the instrument 110 removes tissue (e.g., bone from the vertebrae 2640, 2644, intervertebral disc 2630, etc.). Surrounding non-targeted tissue can be viewed to ensure that the instrument tip 2670 does not injure it. This allows a physician to remove tissue without damaging nerve tissue, the spinal cord, and other non-targeted tissue. The instrument 2610 can have irrigation channels to circulate fluid through the working space to help remove blood, lose tissue, and other anatomical features that may obscure viewing.

Referring to FIGS. 26A-27B, the visualization instrument 140 can be a steerable to facilitate navigation around anatomical features. The visualization instrument 140 can include a fiber-optic scope or a flexible or rigid instrument with one or more illumination elements (e.g., fiber optics for illumination) or imaging elements (e.g., charge-coupled devices for imaging) suitable for visualizing the interior of otherwise inaccessible sites. In some embodiments, the visualization device can be rod-lens endoscopes with an outer diameter equal to or smaller than about 2 mm, 3 mm, 4 mm, 5 mm, 6 mm, 8 mm, or 10 mm, and a length equal to or shorter than about 15 cm, 20 cm, 30 cm, or 40 cm. The visualization instruments 140 can also have integrated irrigation features (e.g., valves, flow control buttons, fluid lumens, return lumens), connectors (e.g., electrical connectors, fluidic connectors, etc.), access ports (e.g., access ports connected to lumens (e.g., lumens through which instruments can pass)), or the like. In embodiments with an angled lens, the visualization instrument can have approximately 0-degree, 10-degree, 15-degree, 30-degree, or 45-degree lens angles, which are toward a light source. In other angled lens embodiments, the visualization instrument can have an approximately 15-degree, 30-degree, or 45-degree lens angled away from a light source. The angle of the lens can be selected based on the area to be viewed. In some posterior or lateral spinal procedures, a 0-degree lens can provide a wide-angle view suitable for viewing nerve roots, the spinal cord, and intervertebral space. A 30- or 45-degree lens endoscope angled toward the light source can be used to provide an angled view toward, for example, the spine or midsagittal plane to view, for example, the spinous processes, spinal cord, or central regions of the intervertebral space. A 30- or 45-degree lens endoscope angled away from the light source can be used to provide an angled view toward the lateral features or the spine, such as nerve roots at the neural foramen, side regions of the intervertebral space, or the like.

### D. TRIANGULATION GUIDES AND TECHNIQUES

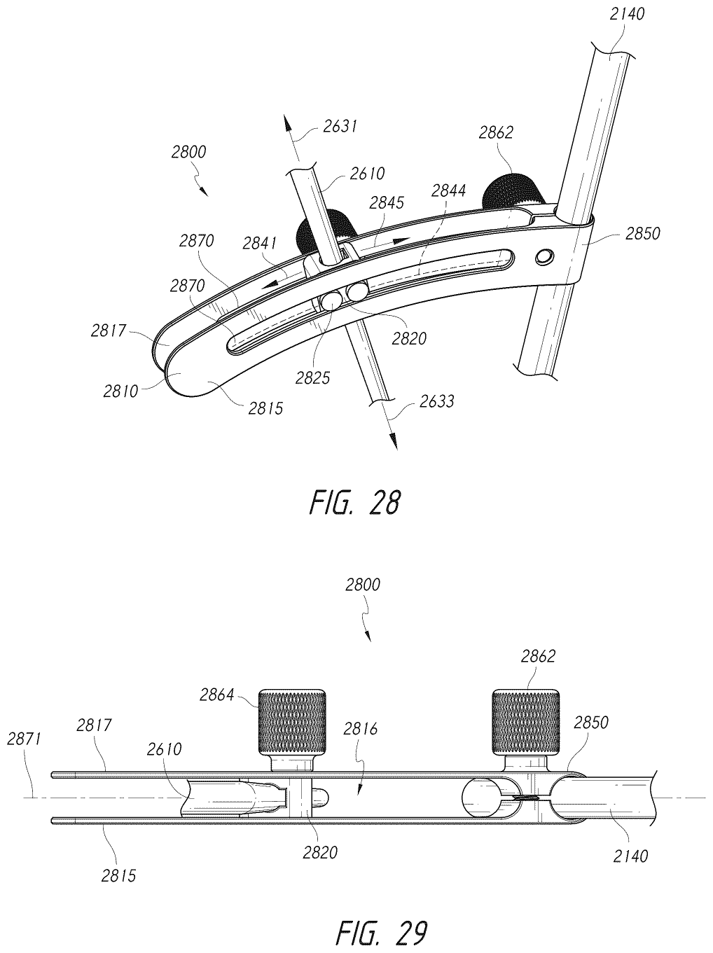

Multi-portal instrument holders can be triangulation guides configured to provide a desired number of degrees of freedom and can be configured to hold multiple instruments to assist with triangulation positioning of the instruments by, for example, limiting their movement (e.g., movement relative to one another, movement relative to the patient, range of motion, etc.). Example triangulation guides and surgical techniques are discussed in connection with FIGS. 28-43.

FIG. 28 is an isometric view of a curved triangulation guide 2800 holding instruments 2610, 2140 in accordance with an embodiment of the disclosure. The curved triangulation guide 2800 can maintain a triangulation relationship between the instruments 2610, 2140 while allowing the instruments 2610, 2140 to be positioned along a generally arcuate path 2844. The instrument 2610 can be moved along the arcuate path 2844, as indicated by arrows 2841, 2845 while the distal ends of instruments 2140, 2610 are, for example, angled toward one another, directed toward a common point (or working space), etc. The arcuate path 2844 can be generally orthogonal to one or both of the longitudinal axes of the instruments 2610, 2140.

The triangulation guide 2800 can include a guide body 2810, curved openings or slots 2870 (“slots 2870”), and one or more instrument holders or clamps 2820 configured to hold instruments (e.g., illustrated clamp 2820 is holding the instrument 2610). The slots 2870 can define one or more travel paths for moving at least one of the instruments 2610, 2140 relative to the other. The instruments 2610, 2140 can be moved away from or toward one another while maintaining triangulation of the instruments 2610, 2140. The instrument clamp 2820 can slide along the opposing slots 2870 while keeping the instrument 2610 aligned or positioned along an imaginary plane (e.g., a center or mid plane 2871 of FIG. 29), a reference line, etc. The curved triangulation guide 2800 can limit the range of motion of the instruments 2610, 2140 to keep distal ends of the instruments along a target path or within target region. The guide body 2810 and/or the slot 2870 can be arcuate (illustrated), wavy, serpentine, and/or another shape suitable for define desired instruments paths. In some embodiments, multiple instrument clamps can be slid along the slots 2870 to position three or more instruments.

FIG. 29 is a top view of the curved triangulation guide 2800. The guide body 2810 can include a front wall 2815 and a back wall 2817 (collectively referenced as “sidewalls 2815, 2817”), and a window or gap 2816 therebetween. The instrument 2610 can be translated along the gap 2816 while the triangulation guide 2800 keeps the instruments 2140, 2610 positioned generally along the imaginary plane 2871. In some embodiments, the instruments 2140, 2610 can move slightly with respect to one another (e.g., away from the plane 2871) to increase the range of motion of the instruments.

Referring now to FIGS. 28 and 29, the instrument clamp 2820 can include a slider 2825 (FIG. 28) that has one or more pins, rollers, and/or wheels that are positioned in the slots 2870. The instrument clamp 2820 can include a locking mechanism 2864 (FIG. 29) that can be used to grip the instrument 2610 while the slider 2825 slides along the slot 2870. The locking mechanism 2864 can be unlocked to, for example, replace the instrument 2610. In some embodiments, the locking mechanism 2864 is used to lock the slider 2825 to the guide body 2810. The slider 2825 can also operate similar to the sliders 712 and 714 described with reference to FIGS. 7 and 8. The instrument clamp 2850 can grip the instrument 2140. The instrument clamp 2850 can include a locking mechanism 2862 generally similar to the locking mechanism 2864 of the instrument clamp 2820. Additionally or alternatively, the instrument clamps 2850 and/or 2820 can be any one of the clamps described in more detail with reference to FIGS. 16-21.

If the curved triangulation guide 2800 is locked, the relative position of the instruments 2140, 2610 can be maintained. For example, the instruments 2140, 2610 can be access tubes or cannulas that are held generally stationary while working instruments, imaging equipment, or tools are delivered through the instruments 2140, 2610. If the triangulation guide 2800 is unlocked, the user can adjust the relative positions of the instruments 2140, 2610. This allows for flexibility when repositioning the instruments for different surgical steps. The instruments can also be moved upwardly or downwardly, as indicated by arrows 2631, 2633 (FIG. 28) for movement of the instrument 2610.

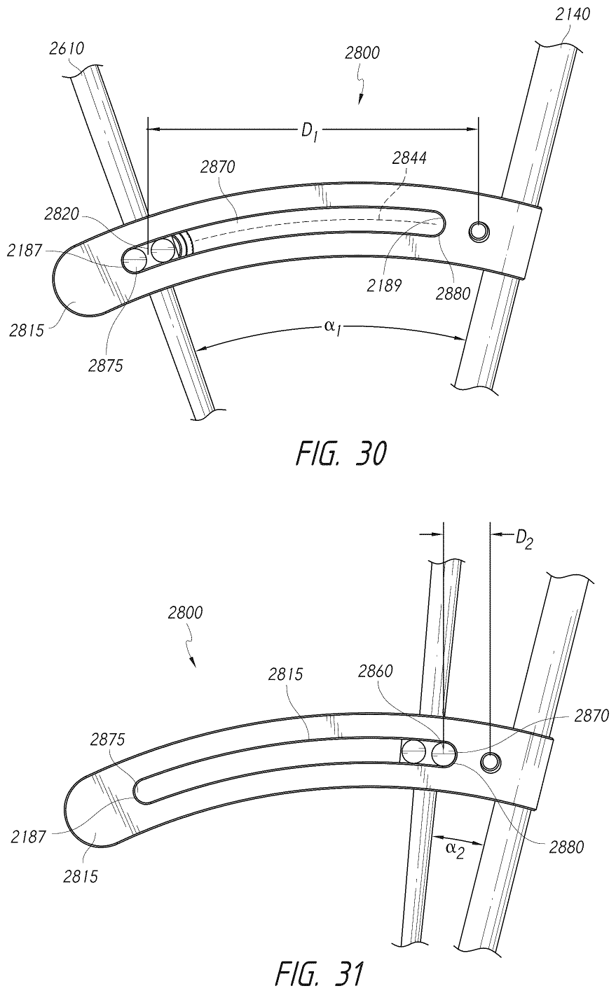

Referring now to FIGS. 28 and 30, the sidewalls 2815, 2817 can have arcuate shapes generally matching the shape of the arcuate path 2844 and/or slots 2870. In addition, the sidewalls 2815, 2817 can include, without limitation, one or more positioning-assist features (e.g., notches, labels, grooves, magnets, or combinations thereof) to help a user determine the location of the instrument 2610 based upon tactile feedback, visual inspection, or the like. The configuration and features of side walls are described in more detail with reference to FIGS. 3-8.

Referring now to FIG. 30, the instruments 2610, 2140 are angled toward one another at an angle α₁ and at distance D₁ (e.g., a distance between the retained portions of the instruments 2610, 2140 or instrument portions located along the path 2844). Referring now to FIGS. 30 and 31, the instrument 2610 can be slid between opposing ends 2187, 2189 of the slots 2870 to position the instrument 2610 further from or closer to the visualization instrument 2140. The instrument 2610 can be locked into place at any position along the slot 2870; therefore, allowing the instruments 2610 and 2140 to be positioned at a range of angular orientations (e.g., 10 degrees, 20 degrees, 30 degrees, 10-30 degrees, etc.) and a range of distances from one another. As shown in FIG. 31, the instruments 2610, 2140 can be positioned next to one another at an angle α₂ and a distance D₂.

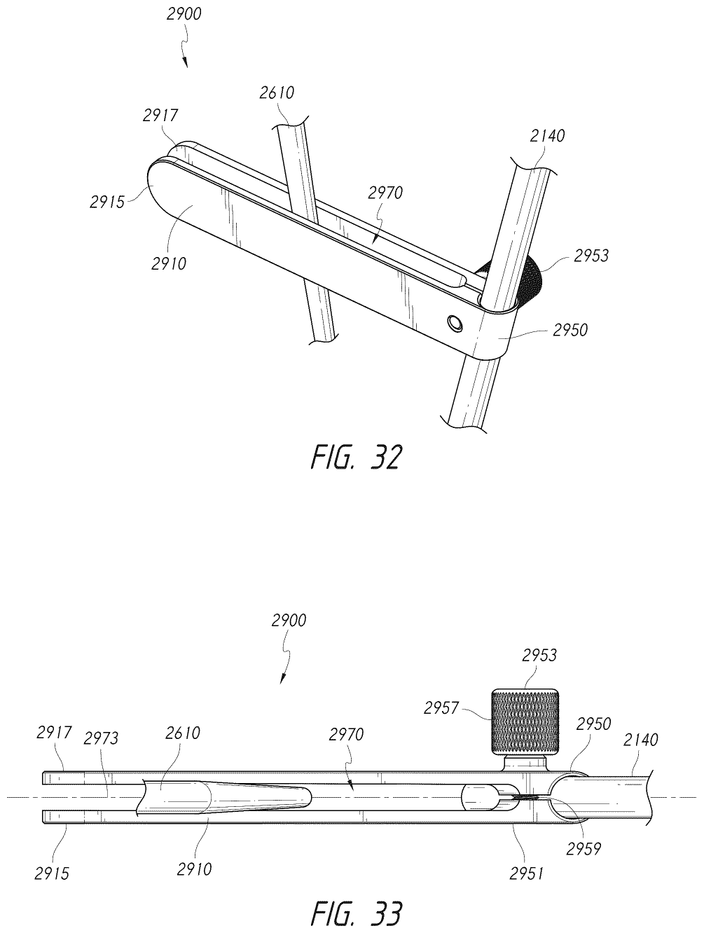

FIGS. 32 and 33 show a linear or straight triangulation guide 2900 holding instruments 2140, 2610 in accordance with an embodiment of the disclosure. The triangulation guide 2900 can include a guide body 2910 and a receiving window or gap 2970 (“gap 2970”). Similar to the triangulation guide 2800 described in connection with FIGS. 28-31, the triangulation guide 2900 can help position the instruments 2140, 2610. The instruments 2610, 2140 can be moved away from or toward one another while maintaining triangulation of the instruments 2610, 2140. For example, the guide body 2910 can be configured to allow movement of the instrument 2610 along the gap 2970 while keeping the instrument 2610 aligned or positioned along an imaginary plane (e.g., a midplane 2973 of the triangulation guide 2900 shown in FIG. 33), a reference line, etc.

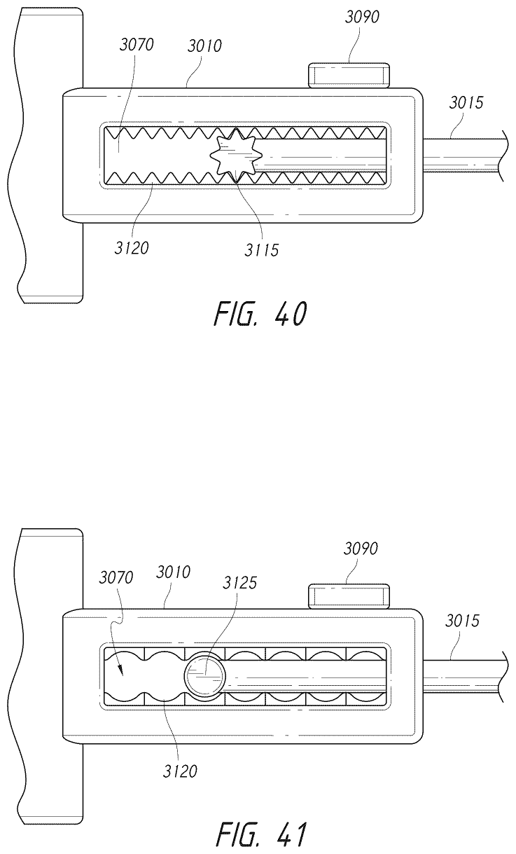

The straight triangulation guide 2900 can include an instrument clamp 2950 generally similar in configuration and in operation to the instrument clamp 2850 of FIGS. 28-31. The instrument clamp 2950 can include a jaw 2951 and an actuation element 2953. The actuation element 2953 can include a threaded body 2959, which threadably engages the jaw 2951, and a handle or knob 2957. The knob 2957 can be rotated to narrow or close the straight triangulation guide 2900 to clamp and/or hold the position of the instrument 2610 without an additional instrument clamp. The guide body 2910 can including cantilevered arms in the form of a front wall 2915 and a back wall 2917. The front wall 2915 and/or back wall 2917 can include one or more retention members (not illustrated) such as teeth, grooves, compressible members, serrated portions, or the like to maintain the position of the instrument 2610 within the gap 2970. Example retention members are described in more detail with reference to FIGS. 40 and 41.

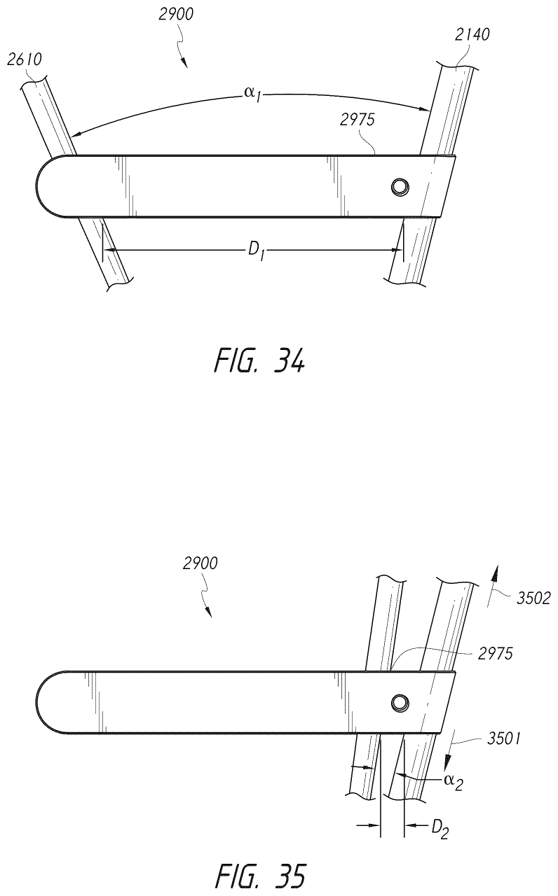

FIGS. 34 and 35 show the instrument 2610 positioned at opposite ends of the gap 2970 (shown in FIGS. 32 and 33) to set a generally greater distance between the two instruments (shown in FIG. 34) or a generally smaller distance between the two instruments (shown in FIG. 35). The instrument 2610 can be slid to an end 2975 of the gap 2970 to position the instrument 2610 closer to the visualization instrument 2140. Additionally or alternatively, the instrument 2140 can be slid (indicated by arrows 3501, 3502 of FIG. 35) along the tubular shaped jaws of the clamp 2950 to adjust the depth of the instrument 2140 in the patient. The instruments 2610, 2140 can then be locked in place using the actuation element 2953 as described in FIG. 33.

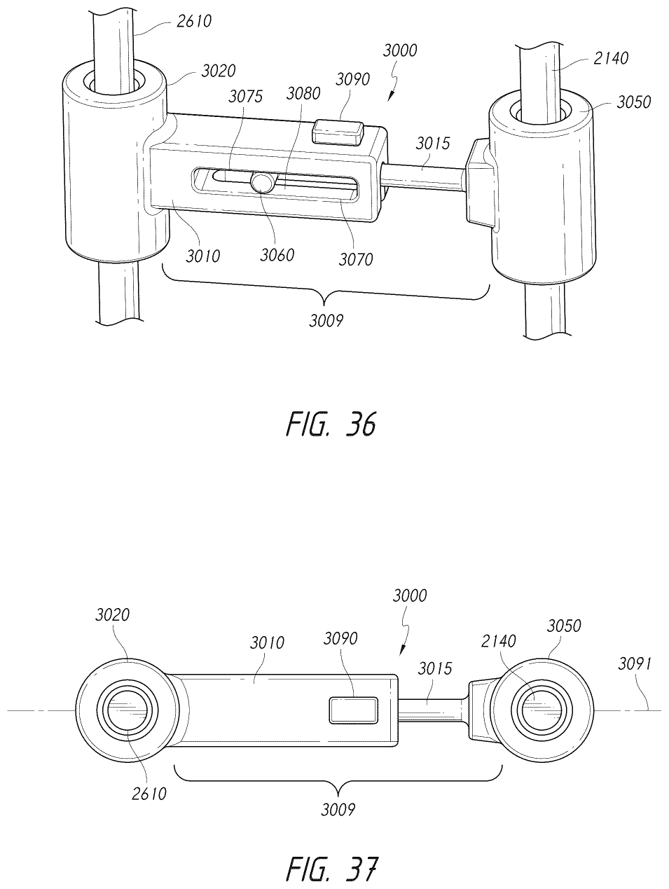

FIG. 36 is an isometric view of an articulatable triangulation guide 3000 in accordance with an embodiment of the disclosure. The triangulation guide 3000 can include a selectively lockable articulating guide joint 3009 including a guide body 3010 and a connector 3015. Instrument holders or clamps 3050, 3020 can be coupled to the connector 3015 and the guide body 3010, respectively. The instrument clamps 3050, 3020 can be generally similar in configuration and in operation to the instrument clamps described in more detail with reference to FIGS. 29 and 33. The connector 3015 (e.g., a T-shaped connector) can be slid along a slot 3070 of the guide body 3010 and can include a pin or pivot 3060 retained within the slot 3070. The slot 3070 can define one or more travel paths for moving at least one of the instruments 2610, 2140 relative to the other. The instruments 2610, 2140 can be moved away from or toward one another while maintaining triangulation of the instruments 2610, 2140. With continued reference to FIG. 36, the guide body 3010 can include one or more track portions 3075, 3080 positioned on opposite sides (e.g., top and bottom) of the slot 3070. As shown in FIGS. 36, 38, and 39, track portions 3075, 3080 are configured to slidably contact the pin 3060.

FIG. 37 is a top view of the triangulation guide 3000. Referring now to FIGS. 36 and 37, a release or trigger 3090 of the guide body 3010 can be used to lock and/or unlock the articulating guide joint 3009. When unlocked, the connector 3015 can be translated along the slot 3070, while maintaining alignment and/or positioning of the instruments 2140 and 2610 relative to an imaginary plane (e.g., midplane 3091 of the triangulation guide 3000). When locked, the articulating guide joint 3009 can hold the instruments 2140, 2610 stationary relative to each other.

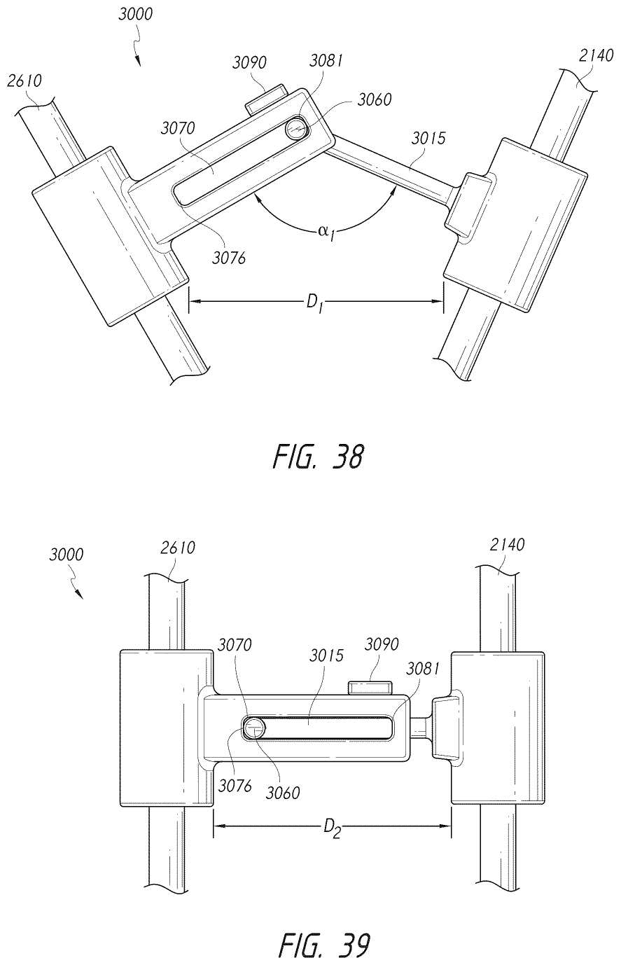

FIGS. 38 and 39 are side views of the triangulation guide 3000 in different configurations. The triangulation guide 3000 can limit the range of motion of the instruments 2140, 2610 to keep distal ends of the instruments along a target path or target region. The articulating guide joint 3009 can be locked (e.g., translationally locked, rotationally locked, or both) with the connector 3015 at any position along the length of the slot 3070 by deploying the trigger 3090, thereby allowing the instruments 2610, 2140 to be positioned at a range of angular orientations and distances from one another. The configuration of the articulating guide joint 3009 can be selected based on the desired adjustability of the instruments (e.g., range of motion), locking (e.g., translation locking, rotational locking, etc.), degrees of freedom, etc.

When the articulating guide joint 3009 is unlocked, the connector 3015 can slide along the slot 3070 until it contacts the slot ends 3076, 3081. As shown in FIG. 38, the pin 3060 of the connector 3015 can be positioned at the slot end 3081 to position the instrument 2610 away from the visualization instrument 2140. Distal ends of the instruments 2610 and 2140 can move toward one another to define an included angle α1 and a distance D₁. FIG. 39 shows the pin 3060 of the connector 3015 located at the slot end 3076 to position the instruments 2610, 2140 next to one another at a distance D₂.

FIGS. 40 and 41 are side views of a joint assembly of the guide body 3010 of the triangulation guide 3000 of FIGS. 38-39 in accordance with some embodiments. The guide body 3010 can include, for example, a rack and pinion locking mechanism (shown in FIG. 40), a clamp locking mechanism (shown in FIG. 41), or lockable joint used to maintain a desired position of the triangulation guide 3000. FIGS. 40 and 41 show the upper and lower portions of slot 3070 configured with retention members 3110, 3120 in the form of teeth and grooves. The retention members 3110, 3120 can also include, for example, compressible members, serrated portions, or the like.

Referring still to FIGS. 40 and 41, the connector 3015 can include a rod or elongated member connected to pivots (e.g., pivots 3115, 3125). The example pivots 3115, 3125 can be configured to contact the retention members 3110, 3120 tightly and lock the triangulation guide 3000 in place. More specifically, the retention members 3110, 3120 can be configured to provide enmeshing, high frictional forces, or other interaction that prevents movement of the pivots 3115, 3125 along the slot 3070. The pivots 3115, 3125 can be positioned at one or more discrete positions within the slot 3070, and the triangulation guide 3000 can then be locked to inhibit, limit, or substantially prevent movement of the connector 3015. To unlock the guide body 3010, a user can depress the trigger 3090 to move apart the retention members 3110, 3120, thereby allow translation/rotation of the connector 3015 along the slot 3070. To lock the guide body 3010, the trigger 3090 can be spring-biased such that the trigger 3090 causes the retention members 3110, 3120 to clamp onto the pivots 3115, 3125.

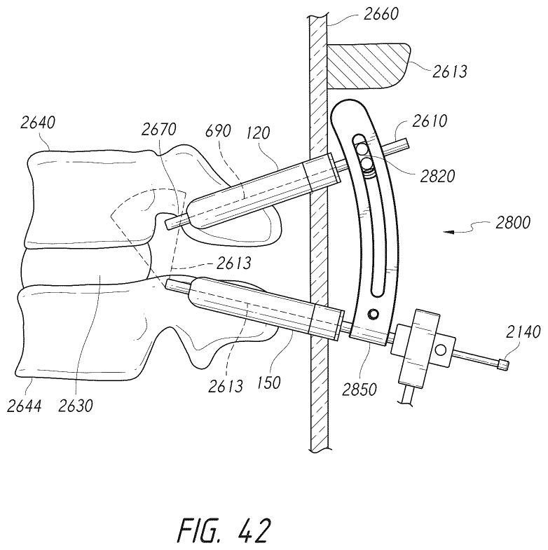

FIG. 42 is a side view of a multi-portal surgical system in accordance with an embodiment of the disclosure. The configuration of the triangulation guide can be selected based on the procedure to be performed. For example, surgical kits can have a set of different configuration triangulation guides that can be selected to perform different procedures, surgical steps, etc. The kit can include instruments designed to be used with the triangulation guides so the user can mix and match instruments and triangulation guides throughout a procedure. Example surgical techniques are described in connection with the triangulation guide 2800, but other surgical guides can be used.

The multi-portal surgical system can include a triangulation guide 2800. Optional cannulas 150, 120 extend through the subject's skin 2660 (thickness not illustrated at scale). The instruments 2140, 2610 can be delivered into an intervertebral space (or other target region or zone) via the cannulas 150, 120. The instrument clamps 2850, 2820 can hold the proximal ends of the instruments 2140, 2610. In some embodiments, the instrument 2140 is a visualization instrument used to position and/or view at least a portion of the disc 2630, vertebral bodies 2640, 2644 and/or the distal portion 2670 of the instrument 2610.

In some embodiments, the triangulation guide 2800 can be used to hold and/or position the instruments 2140,2610 at an angle toward a vertebral level or working zone. The triangulated instruments 2140, 2610 can be used to perform any number of actions at the vertebral level or working zone. The instrument clamp 2850 can hold the visualization instrument 2140 at a desired position for visualization while the instrument clamp 2820 holds the instrument 2610. The triangulation guide 2800 can be adjusted any number of times during a procedure. For example, the triangulation guide 2800 can be used at the beginning of a procedure to provide a close-fitting of the instruments 2140, 2610 in the intervertebral space.

The visualization instrument 2140 can be in used combination or substituted with other visualization techniques, such as one or more of fluoroscopy, MRI imaging, CT imaging, direct visualization. In some procedures, multi-modality imaging of the target site can be performed using an external imaging device. The intraoperative imaging can be displayed via one or more digital screens (e.g., endoscopic imaging and fluoroscopy on different screens) in the surgical room. The triangulation guide 2800 can be periodically reconfigured throughout the procedure depending on the surgical step being performed and/or the results of the intraoperative imaging. For example, if the cannulas 150, 120 need to be reset for installation of a spinal implant in the intervertebral space, the triangulation guide 2800 can be removed and reattached to the instruments 2140, 2610 at a distance that is preferred for installation of the implant, as discussed in more detail with reference to FIGS. 28-39.

The triangulation guide 2800 can be swapped out for a different triangulation guide (e.g., a straight triangulation guide, etc.), or the instrument clamps 2850 and/or 2820 can be swapped out for any of the instrument clamps described in FIGS. 16-21. The triangulation guides and/or the instrument clamps can be swapped out depending on the step of the surgical procedure and/or physician's preference. The device can also be repositioned at the end of a surgical procedure to validate the installation of an implant. For example, one or both cannulas 150,120 can be used to hold one or more visualization instruments 2140. The visualization instruments 2140 can allow for visualization of both sides of the vertebral space, the final implantation site, the trajectory of the implantation, and/or the like. An optional pillow 2613 can be used with the triangulation guide 2800. In some embodiments, the triangulation guide 2800 is coupled to the pillow 2613, as discussed in connection with FIGS. 12-14.

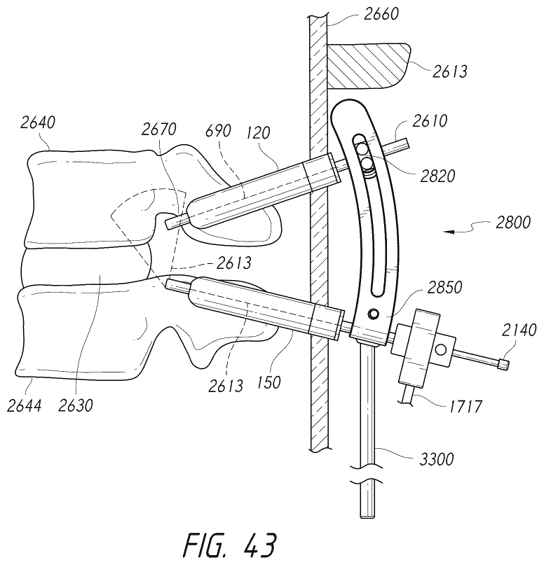

Referring now to FIG. 43, the triangulation guide 2800 can be permanently or detachably coupled to an arm attachment 3300. The arm attachment 3300 can serve as an anchor that maintains the instruments 2140,2610 within a preferred operating window. For example, the arm attachment 3300 can be fixed to a surgical bed and/or external support to maintain the position of the triangulation guide 2800. In some embodiments, a user (e.g., a surgeon, member of the surgical team, etc.) can choose the preferred operating window. For example, the user can choose to anchor the triangulation guide 2800 that sets a maximum allowable rotation of the instruments 2140, 2610. The arm attachment 3300 can maintain the user's preferred operating conditions and can improve outcomes by, for example, preventing the instruments 2140, 2610 from reaching unintended targets (e.g., vital organs, etc.).

The use, number and configuration of the triangulation guides can be selected based on the surgical procedure. For example, the triangulation guide 2800 of FIGS. 42 and 43 can also be used to hold the cannulas 120, 150 rather than the instruments 2140, 2610. For example, the cannulas 120, 150 can be held at a fixed position/relationship to keep working instruments (e.g., distal ends of working instruments) within a targeted working zone. In some procedures, the triangulation guide 2800 holds the cannulas 120, 150 and another triangulation guide 2800 holds the instruments 2140, 2610. This allows for independent positioning of the cannulas 120, 150 and instruments 2140, 2610. The triangulation guide 2800 can maintain triangulation of instruments to keep instruments positioned for bi-portal imaging of working instruments, positioned at the working zone, visualization of the working zone, etc.

The present technology is illustrated, for example, according to various aspects described below as numbered examples (1, 2, 3, etc.) for convenience. These are provided as examples and do not limit the present technology. It is noted that any of the dependent examples may be combined in any combination, and placed into a respective independent example. The other examples can be presented in a similar manner:

1. A triangulation guide for multi-portal procedures, the triangulation guide comprising:
a guide body configured to hold a first instrument; and
a first instrument clamp coupled to the guide body and configured to hold a second instrument;
wherein the first and second instruments are movable relative to one another while held by the guide body and the first instrument clamp, respectively, and
wherein the guide body and the first instrument clamp are configured to maintain triangulation of the first and second instruments.

2. The triangulation guide of example 1, wherein the guide body includes a pair of cantilevered arms defining a gap therebetween for receiving the first instrument.

3. The triangulation guide of example 2, wherein each of the cantilevered arms is arcuate, and wherein the first instrument is movable along a curvature of the pair of cantilevered arms.

4. The triangulation guide of example 2, wherein each of the cantilevered arms is straight.

5. The triangulation guide of example 2, wherein the first instrument clamp comprises a jaw and an actuation element including a threaded body configured to threadably engage the jaw to adjust a width of the gap between the pair of cantilevered arms.

6. The triangulation guide of any one of examples 1-5, further comprising a second instrument clamp coupled to the guide body and configured to hold the first instrument.

7. The triangulation guide of example 6, wherein the second instrument clamp is slidable along a slot of the guide body and detachable from the guide body.

8. The triangulation guide of example 6, wherein the second instrument clamp is fixedly coupled to the guide body.

9. The triangulation guide of any one of examples 1-8, wherein the triangulation of the first and second instruments includes keeping the first and second instruments positioned along an imaginary plane, and wherein the guide body includes a slot defines a travel path for moving at least one of the first or second instrument relative to the other of the first or second instruments.

10. The triangulation guide of any one of examples 1-9, wherein one of the first or second instruments is slidable relative to the guide body to adjust an angular orientation of the first instrument relative to the second instrument between 10-30 degrees.

11. The triangulation guide of any one of examples 1-10, further comprising a connector coupled to the first instrument clamp, wherein the guide body includes an elongated slot configured to slidably receive the connector.

12. The triangulation guide of example 11, wherein the connector includes a pivot rotatably coupled to the elongated slot to allow adjustment of an angular position of the second instrument relative to the first instrument.

13. The triangulation guide of any one of examples 1-12, wherein at least one of the first or second instrument is a cannula.

14. The triangulation guide of any one of examples 1-13, further comprising a locking mechanism configured to lock the triangulation guide to hold the first and second instruments at fixed angular positions relative to one another.

15. The triangulation guide of any one of examples 1-14, wherein the first instrument clamp has a locked configuration to grip the second instrument and unlocked configuration to release the second instrument.

16. A multi-portal method for treating a subject's spine, the method comprising:
inserting a first cannula into a first entrance formed in a subject;
inserting a second cannula into a second entrance formed in the subject, wherein the second entrance is spaced apart from the first entrance; and
positioning a multi-portal holder to hold the first and second cannulas at a fixed relative position while instruments are delivered through the first and second cannulas.

17. The multi-portal method of example 16, further comprising:
setting a distance between the first and second cannulas;
setting an angular orientation between the first and second cannulas; and
locking the multi-portal holder to hold the first and second cannulas at the distance and the angular orientation.

18. The multi-portal method of example 17, further comprising:
unlocking the multi-portal holder;
adjusting at least one of the distance or the angular orientation while the multi-portal holder captively holds the first and second cannulas; and
locking the multi-portal holder to hold the first and second cannulas at the at least one of the distance or the angular orientation.

19. The multi-portal method of any one of examples 16-18, wherein the multi-portal holder includes a triangulation guide configured to hold the first and second cannulas and allow movement of the first cannula along an arcuate path.

20. The multi-portal method of example 19, wherein the triangulation guide has elongated slot and an instrument clamp configured to hold the first cannula, wherein the instrument clamp slides along the slot while the first cannula and the second cannula are positioned generally along an imaginary plane.

21. The multi-portal method of example 19, wherein the triangulation guide comprises one or more instrument holders each configured to hold one of the first and second cannulas or a working instrument.

22. The multi-portal method of example 19, wherein the triangulation guide comprises an articulating guide joint configured to allow articulation of the first and second cannulas while keeping the first and second cannulas positioned generally along an imaginary plane.

23. The multi-portal method of any one of examples 16-22, wherein the multi-portal holder is configured to allow linear translation of the first cannula relative to the second cannula while maintaining radial alignment of the first and second cannula relative to a working space along the subject's spine.

24. The multi-portal method of any one of examples 16-23, wherein the multi-portal holder is configured to allow rotation of the first cannula relative to the second cannula.

25. The multi-portal method of any one of examples 16-24, further comprising: coupling an instrument holder to a surgical bed;
reconfiguring an arm of the instrument holder; and
positioning an instrument in a jaw of the instrument holder, wherein the jaw is coupled to the arm and configured to grip the instrument when the arm is at a fixed configuration for holding the instrument in the subject supported by the surgical bed.

26. A multi-portal method for treating a subject's spine, the method comprising:
inserting a first cannula into a first entrance formed in a subject;
inserting a second cannula into a second entrance formed in the subject, wherein the second entrance is spaced apart from the first entrance; and
positioning a multi-portal holder to hold an instrument at a fixed position relative to the subject while at least one instrument is moved within the first cannula or the second cannula.

27. The multi-portal method of example 26, further comprising:
setting a distance between the instrument and at least one of the first cannula or the second cannula;
setting an angular orientation between the instrument and the at least one of the first cannula or the second cannula; and
locking the multi-portal holder to hold instrument at the setting.

28. A triangulation guide for multi-portal procedures, the triangulation guide comprising:
a guide body configured to hold a first instrument; and
an instrument clamp configured to receive and hold a second instrument, wherein the instrument clamp is slidable along the guide body to adjust a distance between the first and second instruments while keeping the first and second instruments positioned along an imaginary plane.

29. The triangulation guide of example 28 wherein the imaginary plane is a midplane of the guide body.

30. The triangulation guide of example 28 or example 29, wherein the guide body includes spaced apart sidewalls configured to slidably contact the instrument clamp.

31. The triangulation guide of any one of examples 28-30, further including a locking mechanism configured to lock the triangulation guide to hold the first and second instruments at fixed angular positions relative to one another.

32. The triangulation guide of any one of examples 28-31, wherein the guide body includes a slot extending between first and second opposing ends, and wherein the instrument clamp is slidable in the slot between the first and second opposing ends.

33. The triangulation guide of any one of examples 28-32, wherein the guide body is curved, and wherein the instrument clamp is slidable along a curvature of the guide body.

34. The triangulation guide of any one of examples 28-33, wherein the instrument clamp is slidable along the guide body to adjust an angular orientation of the first and second instruments from one another between 10-30 degrees.

35. The triangulation guide of any one of examples 28-34, wherein the instrument clamp has a locked configuration to grip the second instrument and unlocked configuration to release the second instrument.

36. The triangulation guide of any one of examples 28-35, wherein at least one of the first instrument or the second instrument is a cannula.

37. A triangulation guide for multi-portal procedures, the triangulation guide comprising:
a guide body configured to hold a first instrument, wherein the guide body includes a pair of straight cantilevered arms defining a gap therebetween for receiving the first instrument; and
an instrument clamp coupled to the guide body and configured to hold a second instrument, wherein the instrument clamp includes an actuation element configured to adjust a width of the gap.

38. The triangulation guide of example 37, wherein the instrument clamp further includes a jaw, and wherein the actuation element comprises a threaded body configured to threadably engage the jaw to adjust the width of the gap.

39. The triangulation guide of example 38, wherein the jaw is tubular shaped and configured to slidably receive the second instrument.

40. The triangulation guide of any one of examples 37-39, wherein each cantilevered arm includes one or more retention features configured to maintain a position of the first instrument in the gap.

41. A triangulation guide for multi-portal procedures, comprising:
a plurality of instrument holders configured to hold instruments generally along an imaginary plane;
a connector coupled to a first one of the instrument holders; and
a guide body defining an elongated slot configured to hold the connector, wherein the guide body is coupled to a second one of the instrument holders,
   wherein the guide body is configured to allow the connector to slide along at least one slot of the guide body while keeping the plurality of instruments positioned along the imaginary plane.

42. The triangulation guide of example 41, wherein the guide body includes spaced apart sidewalls configured to slidably contact a pivot of the connector.

43. The triangulation guide of example 41 or example 42, wherein the connector includes a pivot rotatably coupled to the at least one slot to allow adjustment of an angular position of distal ends of the instruments.

44. The triangulation guide of any one of examples 41-43, wherein the guide body includes a trigger configured to selectively prevent sliding of the connector along the at least one slot of the guide body.

45. The triangulation guide of any one of examples 41-44, wherein the connector is T-shaped.

46. The triangulation guide of any one of examples 41-45, wherein the guide body includes retention members configured to maintain a position of the connector relative to the guide body.

47. A multi-portal instrument holder, comprising:
a holder body;
a first slider movable along the holder body and including a first clamp configured to receive and hold a first cannula in a first port along a subject; and
a second slider movable along the holder body and including a second clamp configured to receive and hold a second cannula in a second port along the subject.

48. The multi-portal holder of example 47, wherein the holder body has a track along which the first and second sliders are translatable, wherein the first slider includes a first pivoter configured to rotate the first cannula relative to the second cannula.

49. The multi-portal holder of example 47 or example 48, wherein the first slider includes a locking mechanism configured to lock the first instrument at a linear position and/or an angular orientation.

50. An instrument holder comprising:
an arm configured to be coupled to a surgical bed; and
a jaw coupled to the arm and configured to grip an instrument when the arm is at a fixed configuration for holding the instrument in a patient supported by the surgical bed.

51. The instrument holder of example 50, wherein the jaw includes a compressible material facing an opening of the jaw, wherein the compressible material is configured to:
captively hold the instrument positioned in the opening of the jaw when the jaw is in a closed configuration; and
allow the instrument to be removed from the opening when the jaw is in an open configuration.

52. The instrument holder of example 50, wherein the jaw includes spring-loaded grippers facing an opening of the jaw, wherein the spring-loaded grippers are configured to:
captively hold the instrument positioned in the opening of the jaw when the jaw is in a closed configuration; and
allow the instrument to be removed from the opening when the jaw is in an open configuration.

53. A surgical kit comprising:
a first split cannula having a first open channel and a first port flange;
a second split cannula having a second open channel and a second port flange;
a multi-portal holder configured to hold (a) one or both of the first and second split cannulas and/or (b) an instrument positionable through at least one of the first or second split cannula;
at least one support pillow configured to support a position of a user's body manipulating an instrument positioned in the first split cannula and/or the second split cannula; and
a container holding the first and second split cannulas.

54. The surgical kit of example 53, wherein the first split cannula has a first length and the second split cannula has a second length different from the first length.

55. The surgical kit of example 53 or example 54, further comprising a plurality of decompression instruments.

56. A method of forming a surgical support pillow, the method comprising:
determining a position for a surgical instrument or the surgical support pillow relative to a patient;
forming a shape of at least a portion of the surgical support pillow based on the determined position; and
coupling the surgical support pillow to the patient such that the shaped surgical support pillow is configured to support a user holding the surgical instrument in the patient.

57. The method of example 56, wherein the surgical support pillow includes a moldable internal cushion configured to be molded into the shape.

58. The method of example 56 or example 57, wherein the surgical support pillow has a flexible covering configured to contain an internal cushion before and after forming the shape of the surgical support pillow.

59. The method of any one of examples 56-58, wherein the surgical support pillow is configured to be molded to a user's body to become a user-specific disposable support pillow.

60. The method of any one of examples 56-59, wherein the surgical support pillow has an adhesive surface configured to be adhered to a patient's skin.

61. The method of any one of examples 56-60, wherein the surgical support pillow is inflatable.

62. The method of any one of examples 56-61, wherein the surgical support pillow includes a material having a forming state and a formed state.

63. The method of example 62, wherein the forming state includes at least one of a flowable state, an uncured state, or a thermoforming state.

64. The method of example 62, wherein the formed state includes at least one of a non-flowable state, a cured state, or a thermoformed state.

65. The method of any one of examples 56-64, wherein the surgical support pillow is coupled to a multi-portal instrument holder.

66. A surgical support pillow for supporting a user holding a surgical instrument in a patient, comprising:
a moldable region configured to be shaped based on a position of at least one of the surgical instrument or the surgical support pillow relative to the patient.

67. The surgical support pillow of example 66, wherein the moldable region comprises at least one of a thermoformable material, a thermosetting material, a curable material, or a foam.

68. The surgical support pillow of example 66 or example 67, wherein the moldable region is configured to be molded to the user's body such that the surgical support pillow comprises a user-specific, disposable surgical support pillow.

69. The surgical support pillow of any one of examples 66-68, wherein the moldable region is inflatable.

70. The surgical support pillow of any one of examples 66-69, further comprising a flexible configured to contain the moldable region and the non-formable region before and after shaping the moldable region.

71. The surgical support pillow of any one of examples 66-70, further comprising an adhesive pad configured to adhere to the patient's skin or a surgical surface.

72. The surgical support pillow of any one of examples 66-71, wherein the moldable region has a forming state including at least one of a flowable state, an uncured state, or a thermoforming state.

73. The surgical support pillow of any one of examples 66-72, wherein the moldable region has a formed state including at least one of a non-flowable state, a cured state, or a thermoformed state.

74. The surgical support pillow of any one of examples 66-73, wherein the surgical support pillow is coupled to a multi-portal instrument holder.

75. The surgical support pillow of any one of examples 66-74, further comprising a non-formable support region coupled to the moldable region.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof

The above detailed descriptions of embodiments of the technology are not intended to be exhaustive or to limit the technology to the precise form disclosed above. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. Features from various systems, methods, and instruments can be combined with features disclosed in U.S. application Ser. No. 15/793,950; U.S. application Ser. No. 17/902,685; U.S. application Ser. No. 18/335,737; U.S. application Ser. No. 18/464,949; U.S. application Ser. No. 18/470,140; U.S. application Ser. No. 18/596,610; U.S. Pat. Nos. 8,632,594; 9,308,099; 10,105,238; 10,201,431; 10,898,340; 11,464,648; 11,950,770; 9,820,788; 10,799,367; 10,322,009; PCT App. No. PCT/US20/49982; PCT App. No. PCT/US22/21193; PCT App. No. PCT/US21/63881; PCT App. No. PCT/US22/19706; PCT App. No. PCT/US22/21193; PCT App. No. PCT/US24/18567; PCT App. No. PCT/US23/81937; PCT App. No. PCT/US12/58968; and PCT App. No. PCT/US15/43109, which are hereby incorporated by reference and made a part of this application. All patents and patent applications referenced herein are incorporated by reference in their entireties. Variations of the implants are contemplated.

Systems, components, and instruments disclosed herein can be disposable or reusable. For example, the ports, instruments, or cannulas can be disposable to prevent cross-contamination. As used herein, the term “disposable” when applied to a system or component (or combination of components), such as an instrument, a tool, or a distal tip or a head, is a broad term and generally means, without limitation, that the system or component in question is used a finite number of times and is then discarded. Some disposable components are used only once and are then discarded. In other embodiments, the components and instruments are non-disposable and can be used any number of times. In some kits, all of the components can be disposable to prevent cross-contamination. In some other kits, components (e.g., all or some of the components) can be reusable. Various systems, methods, and techniques described above provide a number of ways to carry out the invention. Of course, it is to be understood that not necessarily all objectives or advantages described may be achieved in accordance with any particular embodiment described herein and may depend on the procedures to be performed, robotic system, and end effectors to be used. The robotic system can be used to perform the procedures disclosed herein. The robotic system can include one or more joints, links, grippers (e.g., cannula grippers, instrument grippers, etc.), motors, and effector interfaces, or the like. The configuration and functionality of the robotic system can be selected based on the procedures (including one or more steps) to be performed. A robotic system with a high number of degrees of freedom can be used to perform complicated procedures whereas a robotic system with a low number of degrees of freedom can be used to perform simple procedures. The robotic system can include one or more cameras, imaging devices, computing systems, controllers, and/or displays.

Where the context permits, singular or plural terms may also include the plural or singular term, respectively. Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the present technology. Accordingly, the disclosure and associated technology can encompass other embodiments not expressly shown or described herein.

1. A triangulation guide for multi-portal procedures, the triangulation guide comprising:
a guide body configured to hold a first instrument; and
a first instrument clamp coupled to the guide body and configured to hold a second instrument;
wherein the first and second instruments are movable relative to one another while held by the guide body and the first instrument clamp, respectively, and
wherein the guide body and the first instrument clamp are configured to maintain triangulation of the first and second instruments.

2. The triangulation guide of claim 1, wherein the guide body includes a pair of cantilevered arms defining a gap therebetween for receiving the first instrument.

3. The triangulation guide of claim 2, wherein each of the cantilevered arms is arcuate, and wherein the first instrument is movable along a curvature of the pair of cantilevered arms.

4. The triangulation guide of claim 2, wherein each of the cantilevered arms is straight.

5. The triangulation guide of claim 2, wherein the first instrument clamp comprises a jaw and an actuation element including a threaded body configured to threadably engage the jaw to adjust a width of the gap between the pair of cantilevered arms.

6. The triangulation guide of claim 1, further comprising a second instrument clamp coupled to the guide body and configured to hold the first instrument.

7. The triangulation guide of claim 6, wherein the second instrument clamp is slidable along a slot of the guide body and detachable from the guide body.

8. The triangulation guide of claim 6, wherein the second instrument clamp is fixedly coupled to the guide body.

9. The triangulation guide of claim 1, wherein the guide body includes a slot defines a travel path for moving at least one of the first or second instrument relative to the other of the first or second instruments.

10. The triangulation guide of claim 1, wherein the triangulation of the first and second instruments includes keeping the first and second instruments positioned along an imaginary plane, and wherein one of the first or second instruments is slidable relative to the guide body to adjust an angular orientation of the first instrument relative to the second instrument between 10-30 degrees.

11. The triangulation guide of claim 1, further comprising a connector coupled to the first instrument clamp, wherein the guide body includes an elongated slot configured to slidably receive the connector.

12. The triangulation guide of claim 11, wherein the connector includes a pivot rotatably coupled to the elongated slot to allow adjustment of an angular position of the second instrument relative to the first instrument.

13. The triangulation guide of claim 1, wherein at least one of the first or second instrument is a cannula.

14. The triangulation guide of claim 1, further comprising a locking mechanism configured to lock the triangulation guide to hold the first and second instruments at fixed angular positions relative to one another.

15. The triangulation guide of claim 1, wherein the first instrument clamp has a locked configuration to grip the second instrument and unlocked configuration to release the second instrument.

16. A multi-portal method for treating a subject's spine, the method comprising:
inserting a first cannula into a first entrance formed in a subject;
inserting a second cannula into a second entrance formed in the subject, wherein the second entrance is spaced apart from the first entrance; and
positioning a multi-portal holder to hold the first and second cannulas at a fixed relative position while instruments are delivered through the first and second cannulas.

17. The multi-portal method of claim 16, further comprising:
setting a distance between the first and second cannulas;
setting an angular orientation between the first and second cannulas; and
locking the multi-portal holder to hold the first and second cannulas at the distance and the angular orientation.

18. The multi-portal method of claim 17, further comprising:
unlocking the multi-portal holder;
adjusting at least one of the distance or the angular orientation while the multi-portal holder captively holds the first and second cannulas; and
locking the multi-portal holder to hold the first and second cannulas at the at least one of the distance or the angular orientation.

19. The multi-portal method of claim 16, wherein the multi-portal holder includes a triangulation guide configured to hold the first and second cannulas and allow movement of the first cannula along an arcuate path.

20. The multi-portal method of claim 19, wherein the triangulation guide has elongated slot and an instrument clamp configured to hold the first cannula, wherein the instrument clamp slides along the slot while the first cannula and the second cannula are positioned generally along an imaginary plane.

21. The multi-portal method of claim 19, wherein the triangulation guide comprises one or more instrument holders each configured to hold one of the first and second cannulas or a working instrument.

22. The multi-portal method of claim 19, wherein the triangulation guide comprises an articulating guide joint configured to allow articulation of the first and second cannulas while keeping the first and second cannulas positioned generally along an imaginary plane.

23. The multi-portal method of claim 16, wherein the multi-portal holder is configured to allow linear translation of the first cannula relative to the second cannula while maintaining radial alignment of the first and second cannula relative to a working space along the subject's spine.

24. The multi-portal method of claim 16, wherein the multi-portal holder is configured to allow rotation of the first cannula relative to the second cannula.

25. The multi-portal method of claim 16, further comprising:
coupling an instrument holder to a surgical bed;
reconfiguring an arm of the instrument holder; and
positioning an instrument in a jaw of the instrument holder, wherein the jaw is coupled to the arm and configured to grip the instrument when the arm is at a fixed configuration for holding the instrument in the subject supported by the surgical bed.

26-75. (canceled)
